(19) United States
(12) Patent Application Publication
(10) Pub. No.: US 2023/0129326 A1
(43) Pub. Date: Apr. 27, 2023

(54) METHODS TO PREVENT SARS-COV-2 INFECTION AND TREAT COVID-19

(71) Applicant: University of Florida Research Foundation, Incorporated, Gainesville, FL (US)

(72) Inventors: David A. OSTROV, Gainesville, FL (US); Leah R. REZNIKOV, Gainesville, FL (US); Michael NORRIS, Gainesville, FL (US); Ashley Nicole BROWN, Orlando, FL (US)

(21) Appl. No.: 17/995,050

(22) PCT Filed: Apr. 6, 2021
(86) PCT No.: PCT/US2021/025980

§ 371 Date: Sep. 29, 2022

Related U.S. Application Data
(60) Provisional application No. 63/006,624, filed on Apr. 7, 2020.
(60) Provisional application No. 63/039,195, filed on Jun. 15, 2020.
(60) Provisional application No. 63/070,124, filed on Aug. 25, 2020.

(51) Int. Cl. A61K 31/135; A61K 31/55; A61K 31/655; A61K 31/5415; A61K 31/495; A61K 31/4402; A61K 31/166; C07K 16/10; A61K 45/06; A61K 39/215

(52) U.S. Cl. CPC A61K 31/135; A61K 31/55; A61K 31/655; A61K 31/5415; A61K 31/495; A61K 31/4402; A61K 31/166; C07K 16/10; A61K 45/06; A61K 39/215; A61K 2039/55566

(57) ABSTRACT

Methods of preventing or treating infection by a SARS-CoV-related betacoronavirus are described. The methods include administering to a patient at risk of being infected by a SARS-CoV-related betacoronavirus or suffering from SARS-CoV-related betacoronavirus-related illness a small molecule drug and/or an antibody that binds to the ACE2-SARS interaction domain of either ACE2 or SARS-CoV-2 spike protein. Also described are vaccines comprising S-protein polypeptides corresponding to the ACE-2 interaction domain.

### CROSS-REFERENCE TO RELATED APPLICATIONS

This application claims the benefit of U.S. Provisional Application No. 63/070,124, filed Aug. 25, 2020, U.S. Provisional Application No. 63/039,195, filed Jun. 15, 2020, and U.S. Provisional Application No. 63/006,624, filed Apr. 7, 2020, each of which is incorporated herein by reference.

### STATEMENT REGARDING FEDERALLY SPONSORED RESEARCH OR DEVELOPMENT

This invention was made with government support under grant numbers R00 HL119560, OD023854 and OD026582, awarded by the National Institutes of Health. The government has certain rights in the invention.

### INCORPORATION OF SEQUENCE LISTING

The sequence listing that is contained in the file named T18131W0001_SequenceListing_ST25, which is 1 kilobyte (as measured in Microsoft Windows®) and was created on Mar. 31, 2021, is filed herewith by electronic submission, and is incorporated by reference herein.

### INTRODUCTION

COVID-19 is a global health crisis caused by the novel coronavirus SARS-CoV-2. COVID-19 affects multiple organ systems and, in severe cases, can lead to acute respiratory distress syndrome, abnormal blood clotting, organ failure, pneumonia, septic shock, pediatric multisystem inflammatory syndrome, and more. Many symptoms are respiratory and include cough, shortness of breath, low blood oxygen saturation, loss of smell and taste, and ground glass opacities in the lungs appearing on chest X-ray or CT scan, among others. Severe cases require hospitalization may cause respiratory failure and death. Among recovered patients, many experience lasting damage to the lungs and require respiratory therapy. These patients are further at risk for additional lung pathologies in the future.

SARS-CoV-2 gains access to airway cells through binding to the angiotensin converting enzyme 2 (ACE2). The ACE2 gene encodes the angiotensin-converting enzyme-2, which has been proved to be the receptor for both the SARS-coronavirus (SARS-CoV) and the human respiratory coronavirus NL63. Recent studies and analyses indicate that ACE2 could be the host receptor for the novel coronavirus 2019-nCoV/SARS-CoV-2.

There are currently no approved therapies or vaccines available for COVID-19. Thus, there is an immediate and critical need for therapeutics to treat and prevent SARS-CoV-2 infection.

### SUMMARY

Described herein are compounds and pharmaceutical compositions containing these compounds for use in treating coronavirus infection, including SARS-CoV-2 infection and COVID-19. Methods of using the compounds and pharmaceutical compositions to treat a subject infected by a coronavirus, suspected of being infected by a coronavirus, or at risk of being in infected by a coronavirus are described. In some embodiments, the compounds, pharmaceutical compositions, and methods are used to treat other viral diseases causing respiratory symptoms.

We have identified small molecule drugs that useful as antiviral therapeutics in preventing and/or treating infection caused by SARS-CoV-related betacoronaviruses (SARS coronavirus). The small molecule antiviral drugs include FDA approved drugs and drugs not currently FDA approved. In some embodiments, the SARS-CoV-related betacoronavirus is SARS-CoV or SARS-CoV-2. An infection caused by a SARS-CoV-related betacoronavirus can be, but is not limited to, COVID-19. In some embodiments, the identified drugs can be used to inhibit SARS-CoV-related betacoronavirus replication and/or infection.

We have identified small molecule drugs that bind human ACE2, and/or other off-targets such as human Sigma receptors, and inhibit SARS-CoV-2 viral replication and/or infection by SARS-CoV-2. The small molecule drugs include FDA approved drugs and drugs not currently FDA approved.

In some embodiments, we have identified small molecule drugs that bind human ACE2, a receptor for SARS-CoV-2 spike (S) protein, and inhibit binding of SARS-CoV-2 to ACE2.

In some embodiments, we have identified small molecule drugs that bind human Sigma receptors and inhibit or reduce SARS-CoV-2 viral replication and/or infection by SARS-CoV-2. The small molecule drugs include FDA approved drugs and drugs not currently FDA approved.

The small molecule drugs may exhibit ACE2 enzymatic inhibitory activity and/or ACE2 ability to inhibit SARS coronavirus spike protein-mediated cell fusion. Although an understanding of mechanism is not required for practice, it is believed that some or all of the small molecule drugs may act by binding to ACE2, binding to a Sigma receptor, inhibiting ACE2-SARS interaction, and/or inhibiting Sigma receptor-SARS interaction. The small molecule drugs may be ACE2 and/or Sigma receptor-binding molecules, or the small molecule drugs may be ACE2-SARS and/or Sigma receptor-SARS interaction inhibitors. The small molecule drugs include FDA approved drugs and drugs not currently FDA approved.

In some embodiments, we have identified regions of ACE2 and SARS-CoV-2 spike (S) protein that are important in binding of SARS-CoV-2 to ACE2. Antibodies directed to these sites in ACE2 and/or SARS-CoV-2 spike protein can be used to inhibit binding of SARS-CoV-2 to ACE2.

The identified small molecules and antibodies can be used to prevent or treat SARS-CoV-2 infection in a subject. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject at risk of infection by SARS-CoV-2. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject that has tested positive for SARS-CoV-2. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject that has been exposed to SARS-CoV-2. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject suspected of having been exposed to SARS-CoV-2. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject at risk of being exposed to SARS-CoV-2. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject suffering from or diagnosed with COVID-19. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject to treat acute lung injury in a subject suffering from coronavirus infection, such as COVID-19.

The identified small molecules and antibodies can be used to prevent or treat SARS-CoV-related betacoronavirus infection in a subject. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject at risk of infection by a SARS-CoV-related betacoronavirus. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject that has tested positive for a SARS-CoV-related betacoronavirus. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject that has been exposed to a SARS-CoV-related betacoronavirus. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject suspected of having been exposed to a SARS-CoV-related betacoronavirus. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject at risk of being exposed to a SARS-CoV-related betacoronavirus. In some embodiments, the described small molecules and antibodies (e.g., ACE2- and/or Sigma receptor-binding molecules, or ACE2-SARS interaction inhibitors and/or Sigma-receptor-SARS interaction inhibitors) are administered to a subject suffering from or diagnosed with a SARS-CoV-related betacoronavirus illness.

In some embodiments, a small molecule drug identified as being useful in the prevention and/or treatment of SARS-CoV-related betacoronavirus infection (e.g., an antiviral inhibitors such as an ACE2-SARS interaction inhibitor) is an FDA approved drug that modulates ACE2 catalytic activity (e.g., inhibitor or activator). In some embodiments, a small molecule drug identified as being useful in the prevention and/or treatment of SARS-CoV-related betacoronavirus infection is an FDA approved drug that modulates or interacts with a Sigma receptor or modulates interaction of a SARS-CoV-related betacoronavirus with a Sigma receptor.

In some embodiments, the small molecule drug (e.g., an antiviral inhibitors such as an ACE2-SARS interaction inhibitor or Sigma receptor-SARS interaction inhibitor) is selected from the group consisting of:

(a) diminazene (Azidin, Berenil, Ganasag, Pirocide, CAS No. 536-71-0),

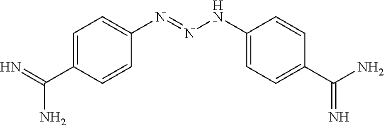 [SMILES: N=C(N)c1ccc(/N=N/Nc2ccc(C(=N)N)cc2)cc1]

(b) diphenhydramine (Benadryl, Nytol, Banophen, etc., CAS No. 58-73-1),

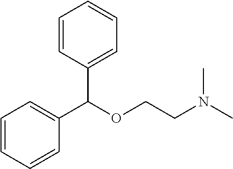 [SMILES: CN(C)CCOC(c1ccccc1)c1ccccc1]

diminazene aceturate salt

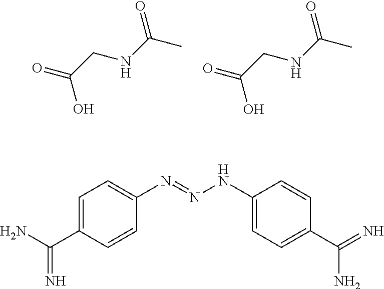

(c) hydroxyzine (ATARAX, VISTARIL CAS No. 68-88-2),

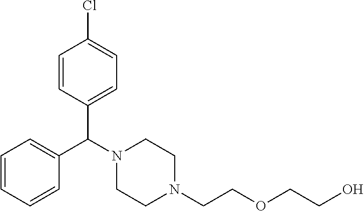 [SMILES: OCCOCCN1CCN(C(c2ccccc2)c2ccc(Cl)cc2)CC1]

(d) cetirizine (ZYRTEC, INCIDAL, CAS No. 83881-51-0),

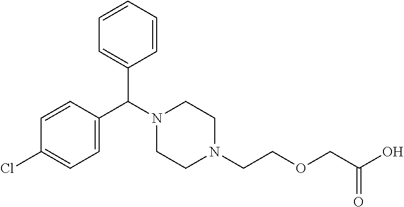 [SMILES: O=C(O)COCCN1CCN(C(c2ccccc2)c2ccc(Cl)cc2)CC1]

L-Stereoisomer, levocetirizine (top) and D-stereoisomer of cetirizine

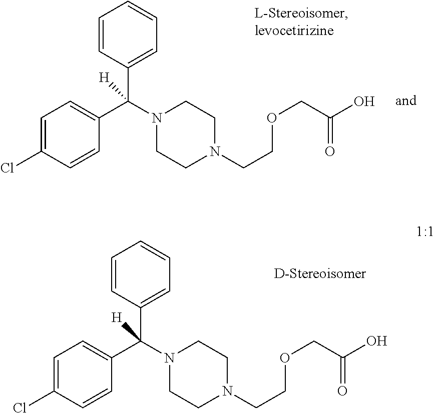 [SMILES: O=C(O)COCCN1CCN([C@H](c2ccccc2)c2ccc(Cl)cc2)CC1 ; O=C(O)COCCN1CCN([C@@H](c2ccccc2)c2ccc(Cl)cc2)CC1]

(e) azelastine (Optivar, Astelin, etc., CAS No. 58581-89-8),

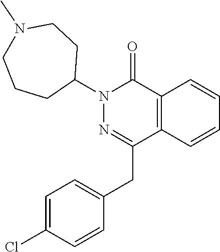 [SMILES: CN1CCCC(n2nc(Cc3ccc(Cl)cc3)c3ccccc3c2=O)CC1]

(f) labetalol (Normodyne, Trandate, CAS No. 36894-69-6),

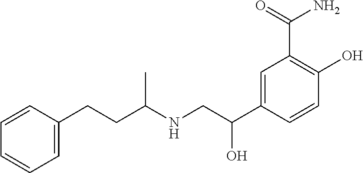 [SMILES: CC(CCc1ccccc1)NCC(O)c1ccc(O)c(C(N)=O)c1]

(g) chlorpheniramine (chlorpheniramine, CAS No. 132-22-9)

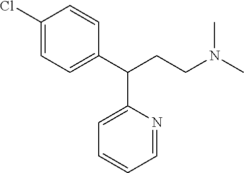 [SMILES: CN(C)CCC(c1ccc(Cl)cc1)c1ccccn1]

(h) aprindine (CAS No. 37640-71-4)

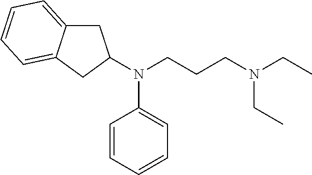 [SMILES: CCN(CC)CCCN(c1ccccc1)C1Cc2ccccc2C1]

(i) minithixen (Chlorprothixene hydrochloride, CAS No. 6469-93-8),

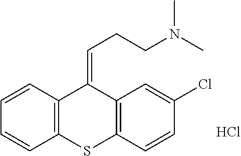 [SMILES: CN(C)CC/C=C1/c2ccccc2Sc2ccc(Cl)cc21 ; Cl]

(j) triethylenetetramine (CAS No. 112-24-3),

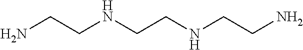

(k) N-(2-aminoethyl)-1-aziridine-ethanamine (CAS No. 23435-23-6),

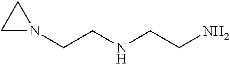

(l) bepotastine (CAS No. 125602-71-3),

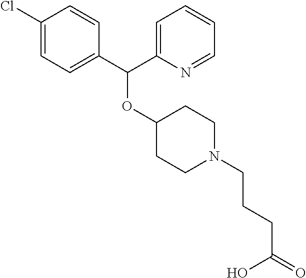 [SMILES: O=C(O)CCCN1CCC(OC(c2ccc(Cl)cc2)c2ccccn2)CC1]

(m) fexofenadine (CAS No. 83799-24-0),

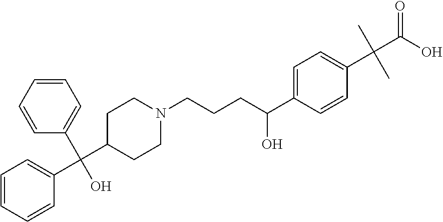 [SMILES: CC(C)(C(=O)O)c1ccc(C(O)CCCN2CCC(C(O)(c3ccccc3)c3ccccc3)CC2)cc1]

(n) loratadine (CAS No. 79794-75-5),

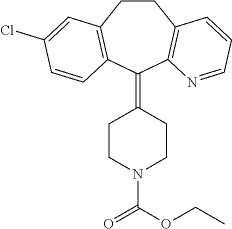 [SMILES: CCOC(=O)N1CCC(=C2c3ccc(Cl)cc3CCc3cccnc32)CC1]

(o) desloratadine (CAS No. 100643-71-8),

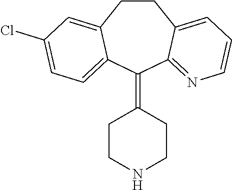 [SMILES: Clc1ccc2c(c1)CCc1cccnc1C2=C1CCNCC1]

(p) ipratropium (CAS No. 22254-24-6 and 60205-81-4),

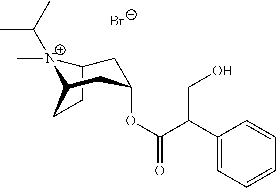

(q) metoclopramide (CAS No. 364-62-5),

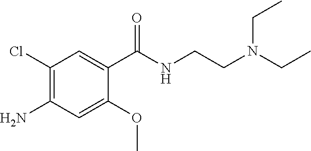 [SMILES: CCN(CC)CCNC(=O)c1cc(Cl)c(N)cc1OC]

(r) domperidone (CAS No. 57808-66-9), and

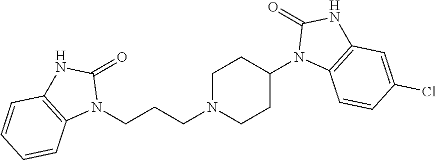 [SMILES: O=c1[nH]c2ccccc2n1CCCN1CCC(n2c(=O)[nH]c3cc(Cl)ccc32)CC1]

(s) myricetin (CAS No. 529-44-2).

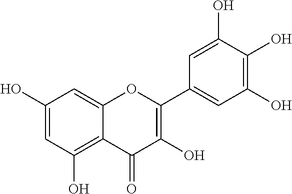 [SMILES: O=c1c(O)c(-c2cc(O)c(O)c(O)c2)oc2cc(O)cc(O)c12]

In some embodiments, an ACE2-SARS interaction inhibitor comprises an anti-SARS-CoV-2 spike protein monoclonal antibody, or an epitope binding fragment thereof, wherein the anti-SARS-CoV-2 spike protein monoclonal antibody, or an epitope binding fragment thereof, binds to one or more amino acids in the region of amino acids 492-503 (LQSYGFQPTNGV (SEQ ID NO: 1)-predicted ACE2-SARS interaction domain) of the SARS-CoV-2 coronavirus spike protein.

In some embodiments, an ACE2-SARS interaction inhibitor comprises an anti-ACE2 monoclonal antibody, or an epitope binding fragment thereof, wherein the anti-ACE2 monoclonal antibody, or an epitope binding fragment thereof, binds to one or more amino acids in the region of amino acids 31-42 (KFNHEAEDLFYQ (SEQ ID NO: 2)-predicted ACE2-SARS interaction domain) of ACE2.

In some embodiments, vaccines against SARS-CoV-2 coronavirus are described comprising peptides comprising or consisting of the amino acid sequence LQSYGFQPTNGV (SEQ ID NO: 1). In some embodiments, vaccines against SARS-CoV-2 coronavirus are described comprising a 9-15 amino acid peptide differing by 0, 1, 2, or 3 amino acid substitutions, deletions, insertions, or combinations thereof from SEQ ID NO: 1. In some embodiments, vaccines against SARS-CoV-2 coronavirus are described comprising a peptide differing by 0, 1, 2, or 3 amino acid substitutions, deletions, insertions, or combinations thereof from SEQ ID NO: 1. In some embodiments, vaccines against SARS-CoV-2 coronavirus are described comprising a peptide having at least 80%, at least 85%, or at least 90% amino acid sequence identity to SEQ ID NO: 1. In some embodiments, vaccines against SARS-CoV-2 coronavirus are described comprising a peptide 12-20, 12-25, 12-30, 12-35, 12-40, 12-45, 12-50, 12-55, 12-60, 12-65, 12-70, 12-75, 12-80, 12-85, 12-90, 12-95, 12-100, 12-125, 12-150, 12-175, 12-200, 12-300, or 12-500 amino acids in length and comprising an amino acid sequence differing by 0, 1, 2, or 3 amino acids from SEQ ID NO: 1. In some embodiments, the peptide is no more than about 12 amino acids in length, no more than about 13 amino acids in length, no more than about 14 amino acids in length, no more than about 15 amino acids in length, no more than about 20 amino acids in length, no more than about 30 amino acids in length, no more than about 50 amino acids in length, no more than about 75 amino acids in length, no more than about 100 amino acids in length, no more than about 200 amino acids in length, no more than about 300 amino acids in length, or no more than about 500 amino acids in length. The vaccines can be used to induce an immune response in a subject to prevent and/or treat SARS-CoV-2 infection.

The isolated polypeptide can be administered by injection. The isolated polypeptide can be formulated for intramuscular injection or intradermal injection. The peptide can be injected into a subject to induce an immune response. The isolated polypeptide can be formulated for injection and for inducing an immune response. The isolated polypeptide can be combined with one or more adjuvants, carriers, excipients, or a combination thereof. In some embodiments, the isolated peptide is linked to a adjuvant. The isolated polypeptide can be combined with a vaccine adjuvant. In some embodiments, the isolated polypeptides is administered with a vaccine adjuvant. In some embodiments, the polypeptide is combined with adjuvant prior to injection. The adjuvant can be, but is not limited to, alum, Sigma Adjuvant System, or Freund's adjuvant. Alum can be, but is not limited to, alum hydrogel.

### BRIEF DESCRIPTION OF THE FIGURES

FIG. 1. Model of spike binding to ACE2 and drugs that prevent binding of SARS-CoV-2 to ACE2. Molecular docking identifies molecules predicted to disrupt binding of SARS-CoV-2 to ACE2.

FIG. 2. Images illustrating diminazene aceturate, a small molecule predicted to decrease binding of SARS-CoV-2 to ACE2, improves airway parameters in a porcine model of airway injury. Airway obstruction in pig airways challenged by acid was mitigated by administration of diminazene aceturate (DZ). (Liao et al. “Acid exposure disrupts mucus secretion and impairs mucociliary transport in neonatal piglet airways.” Am J Physiol Lung Cell Mol Physiol 2020.)

FIG. 3. Graph illustrating NAAE inhibition of SARS S-glycoprotein-mediated cell fusion. 293T cells expressing ACE2 were incubated with NAAE or a control peptide (CP) directed against one of the heptad repeats in the SARS-CoV glycoprotein. Cells were mixed with SARS S1-expressing cells. β-galactosidase activity was used to monitor fusion. *P<0.05 vs DMSO.

FIG. 4. Model illustrating molecular docking that suggests drugs may interact with multiple SARS-CoV-2 targets. Myricetin is a flavonoid predicted to bind the crystal structure of ACE2 (PDB code 1R4L), a model of SARS-CoV-2 helicase based on SARS (6JYT), and the crystal structure of SARS-CoV-2 protease (6LU7). ΔG values output from AutoDock Vina: —9.2 kcal/mol for ACE2, —7.9 kcal/mol for helicase, —7.6 kcal/mol for protease. The predicted ΔG for binding of hydroxyzine to ACE2 was −8.6 kcal/mol (data not shown).

FIG. 5. Model illustrating amino acids at the interface or ACE2 and SARS-CoV-2 spike protein interaction.

FIG. 6. Model representing the closed conformation of ACE2 with drug in the active site.

FIG. 7. Model illustrating binding of Hydroxyzine to ACE2.

FIG. 8. Model illustrating binding of Dextrocetirizine to ACE2.

FIG. 9. Model illustrating binding of Levocetirizine to ACE2.

FIG. 10. Model illustrating binding of Hydroxyzine, Dextrocetirizine, and Levocetirizine to ACE2.

FIG. 11. Graph illustrating inhibition of virus entry into ACE2 expressing airway epithelial cells treated with the indicated therapeutics: loratidine (LOR), diphenhydramine (DIPH), Chlorpheniramine (CHLOR), and azelastine (AZ). n=6 independent experiments. * p=0.0004 DMSO vs DIPH; *p=0.0033 DMSO vs AZ.

FIG. 12. Graph illustrating inhibition of virus entry into ACE2 expressing airway epithelial cells treated with the indicated therapeutics: diminazene (DZ), cetirizine (CET), hydroxyzine (HYD), labetalol (LAB), aprindine (APR), triethylenetetramine (TETA), spike protein (SPK). n=6 independent experiments. *p=0.0063 DMSO vs HYD; *p=0.0032 DMSO vs TETA; *p=0.0011 DMSO vs SPK

FIG. 13. Graph illustrating viral burden in vero6 cells treated with varying concentrations of hydroxyzine.

FIG. 14. Graph illustrating effect of cetirizine (C), diminazene (D), or hydroxyzine (H) on SAR-CoV-2 mRNA expression in infected cells.

FIG. 15A. Graph illustrating inhibition of virus entry into ACE2 expressing airway epithelial cells treated with the indicated therapeutics: hydroxyzine (HYD), diphenhydramine (DIPH), azelastine (AZ), and labetalol (LAB). Numbers after drug name indicate concentrations in μg/ml. n=6 independent experiments. *p=0.0011, DMSO vs HYD 0.1; * p=0.0197, DMSO vs HYD 1.0; *p=0.0124, DMSO vs AZ 0.07; *p=0.0003, DMSO vs AZ 0.7; *p=0.0038, DMSO vs LAB 0.05; p<0.0001, DMSO vs LAB 0.5.

FIG. 15B. Graph illustrating inhibition of virus entry into ACE2 expressing airway epithelial cells treated with the indicated therapeutics: diminazene (DZ), cetirizine (CET), and labetalol (LAB). Numbers after drug name indicate concentrations in μg/ml.

FIG. 16. Graphs illustrating (A) reduction in GFP-expressing SARS-CoV-2 pseudovirus infection of HEK293T treating the DMSO, cetirizine (CET), hydroxyzine (HYD), loratadine (LOR), diphenhydramine (DIPH), azelastine (AZ), or spike protein control (SPK); or (B) reduction in SARS-CoV-2 isolate USA-UF-1/2020 viral plaques (PFUs) present in Vero E6 cells three days post infection in cells treated with HYD, LOR, DIPHEN, CET, or AZ.

FIG. 17. Graphs illustrating dose response curves against SARS-CoV-2 isolate USA-UF-1/2020 in Vero E6 cells for hydroxyzine (A), diphenhydramine (B) and azelastine (C).

FIG. 18. Graph illustrating dose response curve against SARS-CoV-2 isolate USA-WA1/2020 in human lung A549 cells for hydroxyzine.

FIG. 19. Graph illustrating reduction in SARS-CoV-2 replication marker protein in VeroE6 cells infected with SARS-CoV-2 at an MOI of 0.01 following treatment with of azelastine (AZ), diphenhydramine (DIPHEN) or diphenhydramine (PMZ) or combinations thereof.

FIG. 20. Graph illustrating inhibition of interaction of SARS-CoV-2 with ACE2 as determined by competitive ELISA.

FIG. 21. Graph illustrating low viral load in lungs of mice vaccinated with LQSYGFQPTNGV peptide and injected with SARS-CoV-2.

### DETAILED DESCRIPTION

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions such as “not including the endpoints”; thus, for example, “within 10-15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s) or method step(s) that do not materially change the basic and novel characteristics of the compositions and methods described herein may be included in those compositions or methods.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

A “SARS-CoV-related betacoronavirus” or “SARS coronavirus” is a virus that is considered highly similar to or phylogenetically similar to 2003 SARS-CoV or 2019 SARS-CoV-2. A SARS-CoV-related betacoronaviruses can be a betacoronavirus in Lineage B, subgenus Sarbecovirus, or Lineage D, subgenus Nobecovirus. Betacoronaviruses in Lineage A, subgenus Embecovirus (including common human coronaviruses 0C43 and HKU1) and Lineage C, subgenus Merbecovirus (including Middle East respiratory syndrome coronavirus) are not considered SARS-CoV-related betacoronaviruses.

A “homologous” sequence (e.g., nucleic acid sequence or amino acid sequence) refers to a sequence that is either identical or substantially similar to a known reference sequence, such that it is, for example, at least 75%, at least 80%, at least 85%, at least 90%, at least 95%, at least 96%, at least 97%, at least 98%, at least 99%, or 100% identical to the known reference sequence. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

Peptide variants and derivatives are well understood to those of skill in the art and can involve amino acid sequence modifications. An amino acid sequence modification can be a substitution, insertion, or deletion. Insertions include amino and/or carboxyl terminal additions as well as intrasequence insertions of single or multiple amino acid residues. Deletions include the removal of one or more amino acid residues from the peptide sequence. Substitutions include substitution of an amino acid residue at a given position in the amino acid sequence with a different amino acid. Insertions, deletions, and substitutions can occur at a single position or multiple positions. Insertions, deletions, and substitutions can occur at adjacent positions and/or non-adjacent positions. In some embodiments the one or more of the substitutions is a conservative amino acid substitution. Substitutions, deletions, insertions, or any combination thereof may be combined to arrive at a final S-protein polypeptide. For a peptide differing by 0, 1, 2, or 3 amino acids from a reference sequence, the peptide can have substitutions, insertions, or deletions of 0, 1, 2, or 3 amino acids in any combination or order.

“Angiotensin-converting enzyme 2” or ACE2 is an enzyme located on the outer cell membrane of cells in various cell types or organ systems. ACE2 lowers blood pressure by catalyzing the hydrolysis of angiotensin II into angiotensin but also can serve as the entry point for coronaviruses, including SARS CoV 2, into cells.

An “aerosol” is a pharmaceutical composition that is packaged under pressure. Aerosol dosage forms are dispensed when a valve is activated. In some embodiments, aerosol dosage forms can be formulated for application into the nose, or into the lungs by inhalation. Aerosol dosage forms can contain propellants and may be formulated as wet sprays, liquids, powders, or any other form in which inhaled medications are packaged.

As used herein, a “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

An “active pharmaceutical ingredient” is a substance or mixture of substances intended to be used in the manufacture of a drug product and that, when used in the production of a drug, becomes an active ingredient in the drug product. Such substances are intended to furnish pharmacological activity or other direct effect in the diagnosis, cure, mitigation, treatment or prevention of disease or to affect the structure and function of the body.

The term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The phrase “pharmaceutically acceptable” indicates that the substance or composition must be compatible chemically and/or toxicologically, with the other ingredients comprising a formulation, and/or the mammal being treated therewith.

A “subject,” “individual,” or “patient” is vertebrate organism, such as a mammal (e.g., a human).

“Treating” and “treatment” generally refer to obtaining a desired pharmacological and/or physiological effect. In some embodiments, the effect can be, but is not limited to, a prophylactic effect in terms of preventing or partially preventing a disease, or one or more symptoms or conditions associated with coronavirus infection. In some embodiments, the effect can be, but is not limited to, therapeutic effect in terms of a partial or complete cure of a disease, or one or more conditions, symptoms, or adverse effects associated with coronavirus infection or COVID-19.

Recently solved high-resolution crystal structures of the apo-bound and inhibitor-bound forms of ACE2 have provided the basis for a novel molecular docking approach in an attempt to identify ACE2-binding compounds that block SARS coronavirus spike protein-mediated cell fusion. Sequence similarity between SARS-CoV and SARS-CoV-2 suggest that agents (small molecule drugs and antibodies) that bind ACE2 could block binding of a SARS-related coronavirus spike protein, e.g., SARS-CoV-2 spike protein, from binding to ACE2.

We show that specific residues of the SARS-CoV-2 spike protein, including amino acids Gln498, Thr500, and Asn501, form a network of H-bonds with amino acids Tyr41, Gln42, Lys353, and Arg357 of the ACE2 peptidase domain. Using this information, drug candidates were identified through molecular docking analysis (Kulemin et al. “Prediction of off-target effects on angiotensin-converting enzyme 2”. J Biomol Screen 2011, 16:878-885).

139,735 small molecules were screened by in silico molecular docking. In a structure-activity relation study, the molecules with the highest predicted binding scores were identified and assayed for ACE2 enzymatic inhibitory activity and for their ability to inhibit SARS-CoV-2 spike protein-mediated cell fusion. This approach identified N-(2-aminoethyl)-1 aziridine-ethanamine as a novel ACE2 inhibitor that also is effective in blocking the SARS coronavirus spike protein-mediated cell fusion. Thus, the molecular docking approach resulting in the inhibitory capacity of N-(2-aminoethyl)-1 aziridine-ethanamine provides an attractive small molecule compound on which the development of more effective therapeutic agents could be developed to modulate SARS infections.

Using molecular docking analysis, candidate drugs are identified based upon their predicted interactions with the SARS-CoV-2 binding site on ACE2. This approach enables the identification of interactions at multiple sites near the binding site that are likely to create allosteric changes that impair binding of SARS-CoV-related betacoronavirus binding to ACE2.

Although an understanding of mechanism is not required for practice, some of the identified candidates are expected to disrupt SARS-CoV-related betacoronavirus interactions with ACE2. The drugs candidates included both small molecule drugs and monoclonal antibodies that bind to specific regions of ACE2 or the SARS-CoV-2 spike protein.

Because the molecular docking selection strategy was useful in identifying compounds that inhibit or enhance ACE2 catalytic activity, we applied this selection strategy to a chemical library of 1,217 Food and Drug Administration (FDA)-approved compounds with extensive information on bioavailability, toxicity, and safety (Distributed Structure-Searchable Toxicity Database Network, www.epa.gov/ncct/dsstox). Specific FDA-approved compounds that modulate the catalysis by ACE2 were identified.

TABLE 1

| ACE2-Binding Drug | ACE2 binding activity |
|---|---|
| Labetalol ^{a} | Enhances velocity of ACE2 |
| Diminazene ^{a} | Enhances velocity of ACE2 |
| Aprindine ^{a} | Inhibits velocity of ACE2 |
| Minithixen ^{a} | Inhibits velocity of ACE2 |
| Hydroxyzine (Atarax) ^{a} | Inhibits velocity of ACE2 |
| Cetirizine (Zyrtec) ^{a} | Active metabolite of hydroxyzine |
| Triethylenetetramine ^{a} | Inhibits velocity of ACE2 |
| | Inhibits ACE2/SARS spike protein interaction |
| N-(2-aminoethyl)-1 | Inhibits velocity of ACE2 |
| aziridine-ethanamine | Inhibits ACE2/SARS spike protein interaction |

^{a} FDA approved drug

Diphenhydramine, azelastine and hydroxyzine are antihistamines structurally similar to anti-SARS-CoV-2 Sigma receptor-regulating drugs, clemastine and cloperastine (A SARS-CoV-2 protein interaction map reveals targets for drug repurposing. Nature. 2020 July; 583:459-468). In silico molecular docking suggests that diphenhydramine, azelastine and hydroxyzine have the potential to interact with the Sigma-1 receptor and inhibit or reduce SARS-CoV-related betacoronavirus infection.

Hydroxyzine has been shown to exhibit off-target ACE2 activity (Prediction of off-target effects on angiotensin-converting enzyme 2. Kulemina L V, Ostrov D A. J Biomol Screen. 2011 September; 16(8):878-85). Hydroxyzine exhibits anti-SARS-CoV-2 activity with and EC50 of 11.3 μg/ml, and 50% cytotoxic concentration of approximately 50 μg/ml. The known therapeutic window for hydroxyzine indicates hydroxyzine can be successfully used for prevention or treatment of COVID-19.

Diphenhydramine is an over the counter medication with a long safety history. Diphenhydramine is readily available and has a favorable safety profile.

Azelastine is commonly delivered in a nasal formulation.

Inhibitors of host Sigma-1 and Sigma-2 receptors are thought to display antiviral activity by blocking interactions with virus proteins. SARS-CoV-2 NSP6 and ORF9c interact with Sigma receptors implicated in lipid remodeling and the stress response of the endoplasmic reticulum.

In some embodiments, the small molecules and antibodies can be administered to a subject to decrease SARS-CoV-related betacoronavirus disease burden. In some embodiments, the small molecules and antibodies can be administered to a subject to decrease SARS-CoV-related betacoronavirus viral transmission. In some embodiments, the small molecules or antibodies can be administered to a subject to inhibit SARS-CoV-related betacoronavirus infectivity, prevent infection, decrease the likelihood of infection, decrease the severity of infection, and/or decrease the severity or duration of infection. In some embodiments, the small molecules or antibodies can be administered to a subject to inhibit SARS-CoV-related betacoronavirus entry into host cells.

In some embodiments, the small molecules or antibodies (e.g., ACE2-SARS and Sigma receptor-SARS interaction inhibitors) can be used to inhibit SARS-CoV-2 entry into ACE2-expressing host cells, such as, but not limited to, human airway epithelia cells. In some embodiments, disrupting SARS-CoV-2 interactions with ACE2 decreases viral entry into ACE2-expressing cells and decreases viral transmission and/or disease burden.

In some embodiments, the small molecules or antibodies (e.g., ACE2-SARS and Sigma receptor-SARS interaction inhibitors) can be used to inhibit SARS-CoV-related betacoronavirus entry into ACE2-expressing host cells, such as, but not limited to, human airway epithelia cells. In some embodiments, disrupting SARS-CoV-related betacoronavirus interactions with ACE2 decreases viral entry into ACE2-expressing cells and decreases viral transmission and/or disease burden.

In some embodiments, the small molecules or antibodies (e.g., Sigma receptor-SARS interaction inhibitors) bind to host (human) Sigma-1 receptor and prevent interaction of the Sigma receptor with SARS-CoV-2 NSP6 and/or ORF9c.

In some embodiments, the small molecules or antibodies (e.g., Sigma receptor-SARS interaction inhibitors) bind to host (human) Sigma-1 receptor and prevent interaction of the Sigma receptor with SARS-CoV-related betacoronavirus proteins thereby decreasing viral transmission and/or disease burden.

Described are methods of decreasing disease burden, decreasing viral transmission, preventing infection, decreasing the likelihood of infection, decreasing the severity of infection, and/or decreasing the severity or duration of infection. The methods comprise administering one or more of the described small molecules and/or antibodies to a subject that is infected with a SARS-CoV-related betacoronavirus, suspected of being infected with a SARS-CoV-related betacoronavirus, or at risk of being infected with a SARS-CoV-related betacoronavirus.

Described are methods of disrupting SARS-CoV-2 interaction with ACE2 in a subject. Disrupting interaction of SARS-CoV-2 with ACE2 inhibits viral entry and decreases viral transmission and disease burden. In some embodiments, the methods comprise administering one or more of the described small molecules or antibodies to a subject. In some embodiments, the methods comprise administering one or more of the described small molecules or antibodies (e.g., ACE2-SARS interaction inhibitors) to a subject. In some embodiments, the methods comprise administering one or more of the described small molecules or antibodies (e.g., ACE2-SARS interaction inhibitors) to inhibit SARS-CoV-2 entry into human airway cells. In some embodiments, the small molecule or antibody (e.g., ACE2-SARS interaction inhibitor) comprises an FDA-approved small molecule or biologic.

Described are methods of disrupting SARS-CoV-related betacoronavirus interaction with ACE2 in a subject. Disrupting interaction of SARS-CoV-related betacoronavirus with ACE2 inhibits viral entry and decreases viral transmission and disease burden. In some embodiments, the methods comprise administering one or more of the described ACE2-SARS interaction inhibitors to a subject. In some embodiments, the methods comprise administering one or more of the described small molecules or antibodies (e.g., ACE2-SARS interaction inhibitors) to inhibit SARS-CoV-related betacoronavirus entry into human airway cells. In some embodiments, the small molecule or antibody (e.g., ACE2-SARS interaction inhibitor) comprises an FDA-approved small molecule or biologic.

In some embodiments, the described small molecules or antibodies (e.g., ACE2-SARS and Sigma receptor-SARS interaction inhibitors) are administered to a subject at their recognized dosage levels. The standard dosage of diphenhydramine, azelastine and chlorpheniramine were associated with protection from COVID-19.

In some embodiments, the described small molecules or antibodies (e.g., ACE2-SARS and Sigma receptor-SARS interaction inhibitors) are administered according to their recognized administration route.

Formulations and Pharmaceutical Compositions

Any of the described small molecules or antibodies (compounds) or pharmaceutical compositions identified herein for treating coronavirus infection can be formulated as a liquid formulation, as a solid formulation (including a powder or lyophilized formulation), or for aerosol administration. The compounds and pharmaceutical compositions can be formulated as a capsule or tablet, a time-release capsule or tablet, a powder, granules, a solution, a suspension in an aqueous liquid or non-aqueous liquid, an oil-in-water emulsion, or as a water-in-oil liquid emulsion. The compounds and pharmaceutical compositions can be formulated for oral administration, aerosol or inhalation administration, nasal administration, injection, infusion, topical administration, rectal administration, transmucosal administration, transdermal administration, intravenous administration, intradermal administration, subcutaneous administration, intramuscular administration, or intraperitoneal administration. Any of the compounds or pharmaceutical compositions identified herein for treating coronavirus infection can be formulated or packaged for administration by metered-dose inhalers, dry powder inhalers, nebulizers, or soft mist inhalers.

Any of the compounds or pharmaceutical compositions identified herein for treating coronavirus infection can be formulated or packaged in single-dose of multi-dose format.

In some embodiments, any of the compounds or pharmaceutical compositions identified herein for treating coronavirus infection can be formulated for repeat dosing.

Diphenhydramine can be, but is not limited to, a diphenhydramine salt, such as diphenhydramine citrate or diphenhydramine hydrochloride. In some embodiments, diphenhydramine is provided as a liquid formulation, as a tablet, as a coated tablet, as a chewable table, as a powder, or as a capsule. In some embodiments, diphenhydramine is provided in a formulation suitable for transdermal administration, intramuscular administration, or intravenous administration.

In some embodiments, azelastine and/or hydroxyzine are provided in forms suitable for oral administration, such as but not limited to, tablet, a coated tablet, chewable tablet, powder, liquid, or capsule.

In some embodiments, azelastine and/or diminazene are provided in forms suitable for inhalation administration.

A pharmaceutical composition or medicament includes a pharmacologically effective amount of at least one of the described therapeutic compounds (any of the described small molecule or antibodies or pharmaceutically acceptable salts thereof) as an active ingredient, and optionally one or more pharmaceutically acceptable excipients, and optionally one or more additional therapeutic agents. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents and dispersing agents. A carrier may also contain isotonic agents, such as sugars, polyalcohols, sodium chloride, and the like into the compositions.

In some embodiments, two or more drugs may be combined in a single preparation together with pharmaceutically acceptable carriers or diluents, or they may each be present in a separate preparation together with pharmaceutically acceptable carriers or diluents.

In some embodiments, any of the described compounds (small molecules or antibodies) can be combined with one or more additional therapeutic agents. The additional therapeutic agent(s) can be, but are not limited to: an antihistamine, an additional small molecule or antibody as described herein for treating SARS-related betacoronavirus invection, an anti-pruritic, a astringent, a local anesthetic, an interleukin-6 inhibitor such as, for example, sarilumab, siltuximab, tocilizumab, or a combination thereof; an H2 blocker such as, for example, cimetidine, ranitidine, famotidine, nizatidine, roxatidine, lafutidine, lavoltidine, niperotidine, or a combination thereof, an anticoagulant such as, for example, heparin, warfarin, rivaroxaban, dabigatran, apixaban, edoxaban, enoxaparin, fondaparinux, another anticoagulant, or a combination thereof, an antitussive such as, for example, dextromethorphan, benzonatate, or a combination thereof; an antibiotic such as, for example, azithromycin, clarithromycin, doxycycline, levofloxacin, amoxicillin, amoxicillin/clavulanate, or a combination thereof; a glucocorticoid; an interleukin-1 inhibitor such as, for example, anakinra, rilonacept, canakinumab, gevokizumab, or a combination thereof, a cytostatic drug such as, for example, azathioprine, methotrexate, mycophenolate, or a combination thereof, an antiviral agent such as, for example, remdesivir, lopinavir, ritonavir, ribavirin; another cytokine inhibitor or immune modulator such as, for example, infliximab, basiliximab, daclizumab, rapamycin, interferon β-1b, or a combination thereof; and/or a non-steroidal anti-inflammatory drug such as, for example, phenazone, aspirin, celecoxib, diclofenac, ibuprofen, indomethacin, naproxen, oxaprozin, piroxicam, or a combination thereof.

In some embodiments, the small molecule or antibody can be co-formulated with or co-packaged with the additional therapeutic agent(s).

In some embodiments, a pharmaceutical composition or kit comprises azelastine and diphenhydramine. In some embodiments, a pharmaceutical composition or kit comprises diphenhydramine and promethazine.

Kits

In some embodiments, kits containing one or more of the compounds or pharmaceutical compositions identified herein for treating coronavirus infection are described. The kits comprise one or more of the compounds or pharmaceutical compositions identified herein for treating SARS-related betacoronavirus infection and one or more of: (a) at least one agent known to treat a disorder associated with SARS-CoV 2 infection; (b) instructions for treating a disorder associated with SARS-related betacoronavirus infection; or (c) instructions for administering the compound in connection with other betacoronavirus infection therapies.

Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications and/or warnings concerning the use the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

In some embodiments, a kit comprises two or more components, including at least one active pharmaceutical ingredient and one or more inactive ingredients, excipient, diluents, and the like, and optionally instructions for preparation of the dosage form by the patient or person administering the drug to the patient. In some embodiments, a kit may further comprise optional components that aid in the administration of the unit dose to a subject, including but not limited to: vials for reconstituting powder forms, syringes for injection, customized IV delivery systems, inhalers, etc. Additionally, a kit can contain instructions for preparation and administration of the compositions. The kit can be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

In some embodiments, a kit further includes an additional therapeutic agent. The additional therapeutic can be, but it not limited to, an antihistamine, an interleukin-6 inhibitor such as, for example, sarilumab, siltuximab, tocilizumab, or a combination thereof; an H2 blocker such as, for example, cimetidine, ranitidine, famotidine, nizatidine, roxatidine, lafutidine, lavoltidine, niperotidine, or a combination thereof; an anticoagulant such as, for example, heparin, warfarin, rivaroxaban, dabigatran, apixaban, edoxaban, enoxaparin, fondaparinux, another anticoagulant, or a combination thereof; an antitussive such as, for example, dextromethorphan, benzonatate, or a combination thereof; an antibiotic such as, for example, azithromycin, clarithromycin, doxycycline, levofloxacin, amoxicillin, amoxicillin/clavulanate, or a combination thereof; a glucocorticoid; an interleukin-1 inhibitor such as, for example, anakinra, rilonacept, canakinumab, gevokizumab, or a combination thereof; a cytostatic drug such as, for example, azathioprine, methotrexate, mycophenolate, or a combination thereof; an antiviral agent such as, for example, remdesivir, lopinavir, ritonavir, ribavirin; another cytokine inhibitor or immune modulator such as, for example, infliximab, basiliximab, daclizumab, rapamycin, interferon β-1b, or a combination thereof; and/or a non-steroidal anti-inflammatory drug such as, for example, phenazone, aspirin, celecoxib, diclofenac, ibuprofen, indomethacin, naproxen, oxaprozin, piroxicam, or a combination thereof.

Administration

The compounds and pharmaceutical compositions disclosed herein can be administered to a subject once per day, more than once a day, for example, 2, 3, 4, 5 or 6 times a day, or as needed.

In some embodiments, the diphenhydramine is administered to a subject once per day, more than once per day, for example 1, 2, 3, 4, 5, or 6 times per day, or as needed.

In some embodiments, diminazene is administered as an aerosol. In some embodiments, diminazene is administered by nasal administration. By delivering diminazene directly to the lungs, systemic effects from of the drug can be reduced. In some embodiments, diminazene delivered directly to the lungs can exert an immediate and specific potentiating effect on symptoms in the airways. In some embodiments, diminazene can be formulated for aerosol administration once per day, twice per day, or as needed.

In some embodiments, diphenhydramine can be administered orally or parenterally. Parenteral administration can be, but is not limited to, intramuscular administration or intravenous administration. In some embodiments, diphenhydramine is administered orally. In some embodiments, diphenhydramine is administered orally in water or phosphate buffered saline at pH 7.4. In some embodiments, diphenhydramine is administered parenterally.

In some embodiments, an effective amount of diphenhydramine is about 5-600 mg, about 38-468 mg about 25-402 mg, or about 25-300 mg. In some embodiments, an effective amount of diphenhydramine is up 228 mg/day, up to 300 mg/day, up to 400 mg/day, or up to 456 mg/day. In some embodiments, an effective amount of diphenhydramine is about 10-100 mg, about 38-78 mg about 25-67 mg, or about 25-50 mg, administered every orally 4-6 hours. In some embodiments, an effective amount of diphenhydramine is about 10-100 mg or about 10-50 mg administered parenterally. In some embodiments, the diphenhydramine is administered 1 2, 3, 4, 5, or 6 times per day.

In some embodiments, an effective amount of diphenhydramine is about 10-50 mg or about 12.5-25 mg administered 3-4 times/day. In some embodiments, an effective amount of diphenhydramine is about 10 mg, about 12.5 mg, about 25 mg or about 50 mg administered 3-4 times/day. In some embodiments, an effective amount of diphenhydramine is about 5 mg/kg. In some embodiments, an effective amount of diphenhydramine is about 150 mg/m2.

In some embodiments, an effective amount of diphenhydramine is about 19-38 mg every 4-6 hours. In some embodiments, an effective amount of diphenhydramine is about 19 mg or about 38 mg every 4-6 hours. In some embodiments, an effective amount of diphenhydramine is about 38-76 mg every 4-6 hours. In some embodiments, an effective amount of diphenhydramine is about 38 mg or about 76 mg every 4-6 hours. In some embodiments, an effective amount of diphenhydramine is about 6.25 mg every 4-6. In some embodiments, an effective amount of diphenhydramine is about 12.5-25 mg every 4-6 h. In some embodiments, an effective amount of diphenhydramine is about 12.5 mg or about 25 mg every 4-6 h. In some embodiments, an effective amount of diphenhydramine is about 25-50 mg every 4-6 hours. In some embodiments, an effective amount of diphenhydramine is about 25 mg or about 50 mg every 4-6 hours. In some embodiments, an effective amount of diphenhydramine is about 1.25 mg/kg administered up to 4 times per day. In some embodiments, an effective amount of diphenhydramine is about 37.5 mg/m2 administered up to 4 times/day.

In some embodiments, the azelastine is administered to a subject once per day, more than once per day, for example 1, 2, 3, 4, 5, or 6 times per day, or as needed.

In some embodiments, azelastine is administered orally. In some embodiments, azelastine is administered by nasal administration. In some embodiments, azelastine is administered in water or phosphate buffered saline at pH 7.4. In some embodiments, the azelastine is administered as a nasal spray.

In some embodiments, an effective amount of azelastine is about 100-1000 μg, about 137-548 μg, or 205.5-822 μg. In some embodiments, an effective amount of azelastine is about 100-1000 μg/day, about 137-548 μg/day, or about 205.5-822 μg/day. In some embodiments, an effective amount of azelastine is about 137 μg, about 205.5 μg, about 274 μg, about 411 μg, about 548 μg, about 616.5 μg, or about 822 μg.

In some embodiments, the azelastine is administered 1, 2, 3, or 4 times per day. In some embodiments, the azelastine is administered twice per day. In some embodiments, an effective amount of azelastine is about 137-411 μg administered 2 times per day. In some embodiments, an effective amount of azelastine is about 137, about 205.5 μg, about 274 μg, or about 411 μg administered 2 times per day.

In some embodiments, the hydroxyzine is administered to a subject once per day, more than once per day, for example 1, 2, 3, 4, 5, or 6 times per day, or as needed.

In some embodiments, hydroxyzine can be administered orally or parenterally. Parenteral administration can be, but is not limited to, intramuscular administration and intravenous administration. In some embodiments, hydroxyzine is administered orally. In some embodiments, hydroxyzine is administered orally in water or phosphate buffered saline at pH 7.4. In some embodiments, diphenhydramine is administered parenterally

In some embodiments, an effective amount of hydroxyzine is about 25 mg administered 3-4 times/day. In some embodiments, an effective amount of hydroxyzine comprises a single administration of about 50-100 mg administered orally. In some embodiments, an effective amount of hydroxyzine comprises a single administration of about 25-100 mg administered parenterally. In some embodiments, an effective amount of hydroxyzine comprises a single administration of about 25-100 mg administered intramuscularly. In some embodiments, an effective amount of hydroxyzine comprises a single administration of about 0.6 mg/kg administered orally. In some embodiments, an effective amount of hydroxyzine comprises a single administration of about 1.1 mg/kg administered parenterally.

In some embodiments, an effective amount of cetirizine is about 0.5-10 mg, about 1-10 mg, about 2.5-10 mg, about 5-10 mg, about 1-5 mg, or about 2.5-5 mg. In some embodiments, an effective amount of cetirizine is up to 1, 2.5, 5, or 10 mg/day. In some embodiments, an effective amount of cetirizine is about 0.5-5 mg, administered every orally 12-24 hours. In some embodiments, cetirizine is provided in a tablet or in a liquid.

In some embodiments, the pharmaceutical compositions disclosed herein include (or are administered with) H2 blockers. The H2 blocker can be, but is not limited to, cimetidine, ranitidine, famotidine, nizatidine, roxatidine, lafutidine, lavoltidine, niperotidine, and combinations thereof.

In some embodiments, the pharmaceutical compositions disclosed herein include (or are administered with) one or more anticoagulants such as, for example, heparin, warfarin, rivaroxaban, dabigatran, apixaban, edoxaban, enoxaparin, fondaparinux, or a combination thereof.

In some embodiments, the pharmaceutical compositions disclosed herein include (or are administered with) one or more antitussives such as, for example, dextromethorphan, benzonatate, or a combination thereof.

In some embodiments, the pharmaceutical compositions disclosed herein include (or are administered with) one or more antibiotics useful for the treatment of pneumonia including, but not limited to, azithromycin, clarithromycin, doxycycline, levofloxacin, amoxicillin, amoxicillin/clavulanate, and combinations thereof.

In some embodiments, the pharmaceutical compositions disclosed herein include (or are administered with) a glucocorticoid such as, for example, beclomethasone, betamethasone, budesonide, cortisone, dexamethasone, hydrocortisone, methylprednisolone, prednisolone, prednisone, triamcinolone, or a combination thereof.

In some embodiments, the pharmaceutical compositions disclosed herein include (or are administered with) a cytostatic drug such as, for example, azathioprine, methotrexate, mycophenolate.

Antibodies

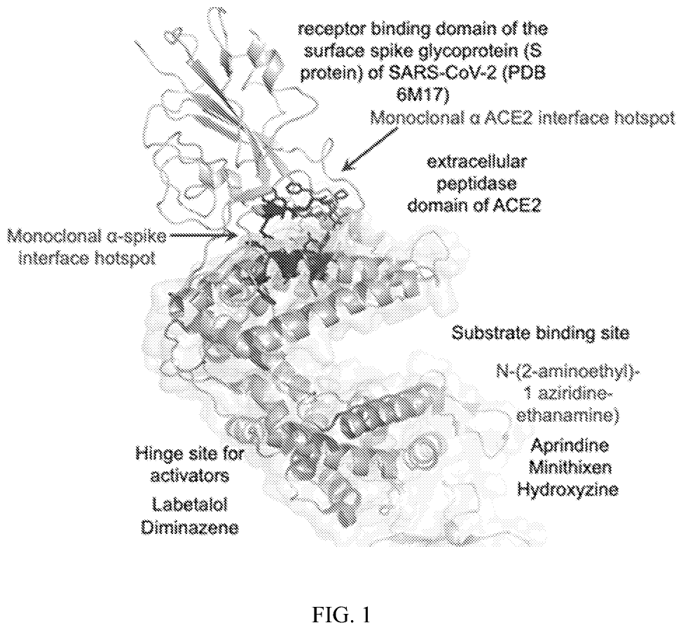

Structural analysis of the interface between SARS-CoV-2 Spike protein and ACE2, based on PDB code 6M17, was used to identify regions of the SARS-CoV-2 spike protein and ACE2 suitable for use as an epitope in generating monoclonal antibodies useful for blocking interaction between the SARS-CoV-2 spike protein and ACE2 (see FIG. 1). The region of interaction corresponded to amino acids 492-503 of the SARS-CoV-2 coronavirus spike protein and amino acids 31-42 of ACE2. Antibodies that bind to the 492-503 region of the SARS-CoV-2 coronavirus spike protein blocked binding of SARS-CoV-2 to ACE2 and acted as neutralizing antibodies in challenge assays. In some embodiments, the monoclonal antibody binds to one or more amino acids in the region of amino acids 492-503 of the SARS-CoV-2 coronavirus spike protein. In some embodiments, the monoclonal antibody binds to one or more amino acids in the region of amino acids 31-42 of ACE2.

In some embodiments, an ACE2-SARS interaction inhibitor comprises an anti-SARS-CoV-2 spike protein monoclonal antibody, or an epitope binding fragment thereof, wherein the anti-SARS-CoV-2 spike protein monoclonal antibody, or an epitope binding fragment thereof, binds to one or more amino acids in the region of amino acids 492-503 (LQSYGFQPTNGV (SEQ ID NO: 1)-predicted ACE2-SARS interaction domain) of the SARS-CoV-2 coronavirus spike protein and inhibits interaction between SARS-CoV-2 spike protein and ACE2. In some embodiments, the anti-LQSYGFQPTNGV (SEQ ID NO: 1) antibodies block binding of SARS-CoV-2 spike protein to ACE2 by at least 50%, at least 60%, at least 70%, at least 80%, or at least 90% when compared to spike protein binding to ACE2 in the absence of the antibodies.

In some embodiments, an ACE2-SARS interaction inhibitor comprises an anti-ACE2 monoclonal antibody, or an epitope binding fragment thereof, wherein the anti-ACE2 monoclonal antibody, or an epitope binding fragment thereof, binds to one or more amino acids in the region of amino acids 31-42 (KFNHEAEDLFYQ (SEQ ID NO: 2)-predicted ACE2-SARS interaction domain) of ACE2, and inhibits interaction between SARS-CoV-2 spike protein and ACE2. In some embodiments, the anti-KFNHEAEDLFYQ (SEQ ID NO: 2) antibodies block binding of SARS-CoV-2 spike protein to ACE2 by at least 50%, at least 60%, at least 70% at least 80%, or at least 90% when compared to spike protein binding to ACE2 in the absence of the antibodies.

Anti-LQSYGFQPTNGV (SEQ ID NO: 1) and anti-KFNHEAEDLFYQ (SEQ ID NO: 2) antibodies can be made using methods known in the art. In some embodiments, the antibodies can be chimeric or humanized.

In some embodiments, methods of generating antibodies that inhibit binding of SARS-CoV-2 from binding to ACE2 are described comprising injecting into a host animal peptide LQSYGFQPTNGV (SEQ ID NO: 1) or peptide KFNHEAEDLFYQ (SEQ ID NO: 2) and isolating antibody producing cells from the injected host animal, identified antibody-producing cells producing antibodies against LQSYGFQPTNGV (SEQ ID NO: 1) or KFNHEAEDLFYQ (SEQ ID NO: 2), and forming hybridoma cells using the identified antibody-producing cells.

Vaccine

Vaccines for preventing or treating SARS-CoV-2 infection are described. In some embodiments, vaccines against SARS-CoV-2 coronavirus are described comprising peptides comprising or consisting of the amino acid sequence LQSYGFQPTNGV (SEQ ID NO: 1). In some embodiments, vaccines against SARS-CoV-2 coronavirus are described comprising a 9-15 amino acid peptide differing by 0, 1, 2, or 3 amino acid substitutions, deletions, insertions, or combinations thereof from SEQ ID NO: 1. In some embodiments, vaccines against SARS-CoV-2 coronavirus are described comprising a peptide differing by 0, 1, 2, or 3 amino acid substitutions, deletions, insertions, or combinations thereof from SEQ ID NO: 1. In some embodiments, vaccines against SARS-CoV-2 coronavirus are described comprising a peptide having at least 80%, at least 85%, or at least 90% amino acid sequence identity to SEQ ID NO: 1. In some embodiments, vaccines against SARS-CoV-2 coronavirus are described comprising a peptide 12-20, 12-25, 12-30, 12-35, 12-40, 12-45, 12-50, 12-55, 12-60, 12-65, 12-70, 12-75, 12-80, 12-85, 12-90, 12-95, 12-100, 12-125, 12-150, 12-175, 12-200, 12-300, or 12-500 amino acids in length and comprising an amino acid sequence differing by 0, 1, 2, or 3 amino acids from SEQ ID NO: 1. In some embodiments, the peptide is no more than about 12 amino acids in length, no more than about 13 amino acids in length, no more than about 14 amino acids in length, no more than about 15 amino acids in length, no more than about 20 amino acids in length, no more than about 30 amino acids in length, no more than about 50 amino acids in length, no more than about 75 amino acids in length, no more than about 100 amino acids in length, no more than about 200 amino acids in length, no more than about 300 amino acids in length, or no more than about 500 amino acids in length. The vaccines can be used to induce an immune response in a subject to prevent and/or treat SARS-CoV-2 infection.

The peptide can be combined with a vaccine adjuvant. In some embodiments, the isolated peptide is administered with a vaccine adjuvant. In some embodiments, the peptide is combined with adjuvant prior to injection. The adjuvant can be, but is not limited to, alum, Sigma Adjuvant System, or Freund's adjuvant. Alum can be, but is not limited to, alum hydrogel.

Described are methods of eliciting an immune response against a SARS-CoV-2 coronavirus. In some embodiments, the methods comprise administering to the subject an effective dose of a peptide comprising, or consisting of SEQ ID NO:1 or a peptide comprising 9-15 amino acid peptide differing by 0, 1, 2, or 3 amino acid substitutions, deletions, insertions, or combinations thereof from SEQ ID NO: 1.

Vaccination can comprise a single administration, or two or more administrations (a prime administration and one or more boost administrations). Prime and boost administrations can be performed at the same site or different sites in the subject, e.g., in the same limb or difference limbs. In some embodiments, the methods comprise administering a first and second dose of the peptide wherein the second dose (boost injection) is administered 2-12 weeks after the first dose (prime injection). In some embodiments, the first and subsequence boost administrations can be performed at intervals of 2-12 weeks. In some embodiments, the first and subsequence boost administrations can be performed at intervals of about 2 about 3, about 4, about 5, about 6, about 7, about 8, about 9, about 10, about 11, or about 12 weeks. In some embodiments, an additional boost is administered about 1-5 years after the prime or boost administration.

Eliciting an immune response against a coronavirus can be used to:

(a) elicit the generation of anti-SARS-CoV-2 antibodies;

(b) elicit the generation of neutralizing anti-SARS-CoV-2 antibodies;

(c) elicit the generation of antibodies protective against SARS-CoV-2 infection;

(d) reduce the likelihood of SARS-CoV-2 infection or lessen the severity of SARS-CoV-2 infection;

(e) reduce the likelihood of developing COVID-19 or lessen the severity or duration of COVID-19;

(f) vaccinate a patient against SARS-CoV-2 or COVID-19;

(g) elicit protective immunity against COVID-19 or severe COVID-19;

(h) prevent at least one symptom of COVID-19 disease, and/or

(i) prevent symptomatic COVID-19 disease.

### Therapeutic Use

In some embodiments, the compounds and pharmaceutical compositions are administered to a subject at risk of infection by a coronavirus, a subject that has tested positive for a coronavirus, a subject that has been exposed to a coronavirus, a subject suspected of having been exposed to a coronavirus, a subject at risk of being exposed to a coronavirus, a subject suffering from or diagnosed with a coronavirus-related condition, or a subject suffering from acute lung injury due to a coronavirus infection.

In some embodiments, the compounds and pharmaceutical compositions are administered to a subject at risk of infection by a betacoronavirus, a subject that has tested positive for a betacoronavirus, a subject that has been exposed to a betacoronavirus, a subject suspected of having been exposed to a betacoronavirus, a subject at risk of being exposed to a betacoronavirus, a subject suffering from or diagnosed with betacoronavirus-related condition, or a subject suffering from acute lung injury due to a betacoronavirus infection.

In some embodiments, the compounds and pharmaceutical compositions are administered to a subject at risk of infection by SARS-CoV-2, a subject that has tested positive for SARS-CoV-2, a subject that has been exposed to SARS-CoV-2, a subject suspected of having been exposed to SARS-CoV-2, a subject at risk of being exposed to SARS-CoV-2, a subject suffering from or diagnosed with COVID-19, or a subject suffering from acute lung injury due to SARS-CoV-2 infection.

Described are methods of reducing the duration of SARS-CoV-related betacoronavirus infection, reducing the severity of SARS-CoV-related betacoronavirus infection, reducing the likelihood of SARS-CoV-related betacoronavirus infection, reducing SARS-CoV-related betacoronavirus replication, or reducing the likelihood of developing a SARS-CoV-related betacoronavirus-related illness in a subject, the method comprising administered to the subject one or more of the described compounds and pharmaceutical compositions. The SARS-CoV-related betacoronavirus can be, but is not limited to, SARS-CoV-2.

The compounds and pharmaceutical compositions can be administered to a subject at risk of infection by a SARS-CoV-related betacoronavirus, a subject that has tested positive for a SARS-CoV-related betacoronavirus, a subject that has been exposed to a SARS-CoV-related betacoronavirus, a subject suspected of having been exposed to a SARS-CoV-related betacoronavirus, a subject at risk of being exposed to a SARS-CoV-related betacoronavirus, or a subject suffering from or diagnosed with SARS-CoV-related betacoronavirus illness. The SARS-CoV-related betacoronavirus can be, but is not limited to, SARS-CoV-2.

Described are methods of reducing the duration of SARS-CoV-2 infection, reducing the severity of SARS-CoV-2 infection, reducing the likelihood of SARS-CoV-2 infection, reducing SARS-CoV-2, or reducing the likelihood of developing COVID-19 in a subject, the method comprising administered to the subject one or more of the described compounds and pharmaceutical compositions.

In some embodiments, the compounds and pharmaceutical compositions described herein can be administered to a subject to decrease SARS-related betacoronavirus entry into cells. In some embodiments, the compounds and pharmaceutical compositions described herein can be administered to a subject to decrease SARS-related betacoronavirus entry into cells via ACE2. The SARS-related betacoronavirus can be, but is not limited to SARS-CoV-2.

In some embodiments, the compounds and pharmaceutical compositions described herein can be administered to a subject protect airways of a subject during infection by a SARS-related betacoronavirus. The SARS-related betacoronavirus can be, but is not limited to SARS-CoV-2.

In some embodiments, the compounds and pharmaceutical compositions described herein can be administered to a subject to decrease viral load or to prevent an increase in viral load in a subject infected with a SARS-related betacoronavirus. The SARS-related betacoronavirus can be, but is not limited to SARS-CoV-2. “Viral load” refers to the amount of virus in a given volume of a body fluid such as blood plasma.

In some embodiments, the compounds and pharmaceutical compositions described herein can be administered to a subject to ameliorate one or more symptoms associated with SARS-related betacoronavirus infection in a subject infected with a SARS-related betacoronavirus or suspected of being infected with a SARS-related betacoronavirus. The symptoms can be, but are not limited to: cytokine storm, lung fibrosis, pulmonary fibrosis, ground glass opacities, pulmonary fibrosis, acute respiratory distress syndrome (ARDS), and/or pneumonia. In some embodiments, the compounds and pharmaceutical compositions described herein can be administered to improve mucociliary transport or mitigate airway obstruction in a subject infected with a SARS-related betacoronavirus or suspected of being infected with a SARS-related betacoronavirus. The SARS-related betacoronavirus can be, but is not limited to, SARS-CoV-2.

In some embodiments, the compounds and pharmaceutical compositions described herein can be administered to a subject to reduce one or more symptoms associated with SARS-related betacoronavirus infection in a subject infected with a SARS-related betacoronavirus or suspected of being infected with a SARS-related betacoronavirus. The SARS-related betacoronavirus can be, but is not limited to, SARS-CoV-2. The symptoms can be, but are not limited to: cytokine storm, lung fibrosis, pulmonary fibrosis, ground glass opacities, pulmonary fibrosis, acute respiratory distress syndrome (ARDS), and/or pneumonia

In some embodiments, the compounds and pharmaceutical compositions described herein can be administered to a subject to prevent or reduce the risk of developing one or more symptoms associated with SARS-related betacoronavirus infection in a subject infected with a SARS-related betacoronavirus, suspected of being infected with a SARS-related betacoronavirus, or at risk of being injected with a SARS-related betacoronavirus. The SARS-related betacoronavirus can be, but is not limited to SARS-CoV-2. The symptoms can be, but are not limited to: cytokine storm, lung fibrosis, pulmonary fibrosis, ground glass opacities, pulmonary fibrosis, acute respiratory distress syndrome (ARDS), and/or pneumonia

In some embodiments, the compounds and pharmaceutical compositions described herein can be administered to a subject to treat a condition caused by SARS-related betacoronavirus infection, prevent a condition cause by SARS-related betacoronavirus infection, or reduce the severity of a condition caused by SARS-related betacoronavirus infection in a subject infected with a SARS-related betacoronavirus, suspected of being infected with a SARS-related betacoronavirus, or at risk of being injected with a SARS-related betacoronavirus. The SARS-related betacoronavirus can be, but is not limited to, SARS-CoV-2. The condition can be, but is not limited to, COVID-19. Treating includes, but is not limited to, reducing the length of time symptoms are experienced, or the like.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### Listing of Embodiments

1. A method of treating a subject suffering from infection by or susceptible to infection by a SARS-CoV-related betacoronavirus comprising administering to a subject a therapeutically effective amount of one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; myricetin; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

2. A method of treating a subject suffering from a SARS-CoV-related betacoronavirus-related illness comprising administering to the subject a therapeutically effective amount of one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; myricetin; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

3. A method of preventing infection by a SARS-CoV-related betacoronavirus comprising administering to a subject a therapeutically effective amount of one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; myricetin; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

4. The method of any one of embodiments 1-3, wherein the agent comprises diphenhydramine and/or azelastine.

5. The method of embodiment 4, wherein the diphenhydramine and/or azelastine comprises diphenhydramine.

6. The method of embodiment 5 wherein the method further comprises administering promethazine to the subject.

7. The method of embodiment 4, wherein the diphenhydramine and/or azelastine comprises azelastine.

8. The method of embodiment 4, wherein the diphenhydramine and/or azelastine comprises a combination of diphenhydramine and azelastine.

9. The method of any one of embodiments 1-3, wherein the agent comprises diminazene.

10. The method of any one of embodiments 1-3, wherein the agent comprises hydroxyzine.

11. The method of any one of embodiments 1-3, wherein the agent comprises cetirizine

12. The method of any one of embodiments 1-3, wherein the agent comprises chlorpheniramine.

13. The method of any one of embodiments 1-3, wherein the agent comprises labetalol.

14. The method of any one of embodiments 1-3, wherein the agent comprises the anti-ACE2 monoclonal antibody, or epitope binding fragment thereof, that binds to one or more amino acids in the region of amino acids 31-42 of ACE2 and inhibits binding of SARS-CoV-2 spike protein to ACE2.

15. The method of any one of embodiments 1-3, wherein the agent comprises the anti-spike protein monoclonal antibody, or epitope binding fragment thereof, that binds to one or more amino acids in the region of amino acids 492-503 of the SARS-CoV-2 coronavirus spike protein and inhibits binding of SARS-CoV-2 spike protein to ACE2.

16. The method of any one of embodiments 1-3, wherein the agent comprises the peptide have the amino acid sequence of SEQ ID NO: 1

17. The method of any one of embodiments 1-13, wherein the method further comprises administering one or more additional therapeutic agents.

18. The method of embodiment 8, wherein the one or more additional therapies comprises an effective amount of at least one drug selected from the group consisting of: one or more agents selected from the group consisting of: diphenhydramine; azelastine; diminazene; hydroxyzine; cetirizine; labetalol; promethazine; aprindine; minithixen; triethylenetetramine; N-(2-aminoethyl)-1-aziridine-ethanamine; bepotastine; fexofenadine; loratadine; desloratadine; ipratropium; metoclopramide; domperidone; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

19. The method of any one of embodiments 1-2 and 4-18, wherein the subject has tested positive for a SARS-CoV-related betacoronavirus, has been exposed to a SARS-CoV-related betacoronavirus, is suspected of having been exposed to a SARS-CoV-related betacoronavirus, is at risk of being exposed to a SARS-CoV-related betacoronavirus, is suffering from or diagnosed with a SARS-CoV-related betacoronavirus-related illness, or is suffering from acute lung injury due to a SARS-CoV-related betacoronavirus-related illness.

20. The method of any one of embodiments 1-19, wherein the SARS-CoV-related betacoronavirus is SARS-CoV-2.

21. The method of any one of embodiments 2 and 4-20, wherein the SARS-CoV-related betacoronavirus-related illness is COVID-19.

22. A SARS-CoV-related betacoronavirus therapeutic comprising an anti-spike protein monoclonal antibody, or epitope binding fragment thereof, that binds to one or more amino acids in the region of amino acids 492-503 of the SARS-CoV-2 coronavirus spike protein and inhibits binding of SARS-CoV-2 spike protein to ACE2.

23. The SARS-CoV-related betacoronavirus therapeutic of embodiment 22, wherein the anti-spike protein monoclonal antibody, or epitope binding fragment thereof, binds the polypeptide LQSYGFQPTNGV (SEQ ID NO: 1).

24. A SARS-CoV-related betacoronavirus therapeutic comprising an anti-ACE2 monoclonal antibody, or epitope binding fragment thereof, that binds to one or more amino acids in the region of amino acids 31-42 of ACE2 and inhibits binding of SARS-CoV-2 spike protein to ACE2.

25. The SARS-CoV-related betacoronavirus therapeutic of embodiment 24, wherein the anti-spike protein monoclonal antibody, or epitope binding fragment thereof, binds the polypeptide KFNHEAEDLFYQ (SEQ ID NO: 2).

26. Use of the SARS-CoV-related betacoronavirus therapeutic of any one of embodiments 22-25 in the treatment of a subject suffering from infection by a SARS-CoV-related betacoronavirus.

27. A method of treating SARS-CoV-2 infection in a subject comprising administering to the subject an antibody, or an epitope binding fragment thereof, specific for LQSYGFQPTNGV (SEQ ID NO: 1) or KFNHEAEDLFYQ (SEQ ID NO: 2)

28. A SARS-CoV-2 vaccine comprising a polypeptide having at least 80% sequence identity to the amino acid sequence of SEQ ID NO: 1 or a peptide comprising 9-15 amino acids differing by 0, 1, 2, or 3 amino acid substitutions, deletions, insertions, or combinations thereof from SEQ ID NO: 1.

29. The SARS-CoV-2 vaccine of embodiment 28, wherein the polypeptide is no more than about 100 amino acids in length.

30. The SARS-CoV-2 vaccine of embodiment 28, wherein the polypeptide amino acid sequence consists of SEQ ID NO: 1.

31. The SARS-CoV-2 vaccine of any one of embodiments 28-30, further comprising an adjuvant.

32. The SARS-CoV-2 vaccine of embodiment 31, wherein the adjuvant comprises: AddaVax, Montanide, alum, Sigma Adjuvant System, or Freund's adjuvant.

33. Use of the vaccine of any one of embodiments 28-32 to induce a protective immune response in a subject to prevent and/or treat a SARS-CoV-2 infection.

34. A method of inducing an immune response to SARS-CoV-2 in a subject comprising: administering to the subject the vaccine of any one of embodiments 28-32.

35. A pharmaceutical composition comprising one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; an anti-spike protein monoclonal antibody, or epitope binding fragment thereof, that binds to one or more amino acids in the region of amino acids 492-503 of the SARS-CoV-2 coronavirus spike protein; and an anti-ACE2 monoclonal antibody, epitope binding fragment thereof, that binds to one or more amino acids in the region of amino acids 31-42 of ACE2, and a peptide have the amino acid sequence of SEQ ID NO: 1, for use in the treatment of a SARS-related betacoronavirus infection.

36. The pharmaceutical composition of embodiment 35 wherein the composition comprises diphenhydramine and azelastine.

37. The pharmaceutical composition of embodiment 35 or embodiment 32 further comprising at least one pharmaceutically acceptable excipient.

38. The pharmaceutical composition of any one of embodiments 35-37, further comprising one or more additional therapeutic agents.

39. The pharmaceutical composition of embodiment 35, wherein the additional therapeutic agent is selected from the group consisting of: an antihistamine, an IL-6 inhibitor, sarilumab, siltuximab, tocilizumab, an H2 blocker, ranitidine, famotidine, nizatidine, roxatidine, lafutidine, lavoltidine, niperotidine, an anticoagulant, heparin, warfarin, rivaroxaban, dabigatran, apixaban, edoxaban, enoxaparin, fondaparinux, an antitussive, dextromethorphan, benzonatate, an antibiotic, azithromycin, clarithromycin, doxycycline, levofloxacin, amoxicillin, clavulanate, an IL-1 inhibitor, rilonacept, canakinumab, gevokizumab, a cytostatic drug, azathioprine, methotrexate, mycophenolate, a cytokine inhibitor, an immune modulator, infliximab, basiliximab, daclizumab, rapamycin, interferon β-1b, an antiviral agent, remdesivir, lopinavir, ritonavir, ribavirin, a glucocorticoid, beclomethasone, betamethasone, budesonide, cortisone, dexamethasone, hydrocortisone, methylprednisolone, prednisolone, prednisone, triamcinolone, a non-steroidal anti-inflammatory drug, phenazone, aspirin, celecoxib, diclofenac, ibuprofen, indomethacin, naproxen, oxaprozin, piroxicam, or a combination thereof.

40. The pharmaceutical composition of embodiment 39 wherein the pharmaceutical composition comprises diphenhydramine and promethazine.

41. The pharmaceutical composition of any of embodiments 35-40, wherein the pharmaceutical composition is formulated for oral delivery, intravenous injection, intramuscular injection, subcutaneous injection, or inhalation.

42. The pharmaceutical composition of embodiment 41, wherein the pharmaceutical composition is dispensed by a metered-dose inhaler, a dry powder inhaler, a nebulizer, or a soft mist inhaler.

43. The pharmaceutical composition of any one of embodiments 35-42, wherein the SARS-related betacoronavirus infection comprises COVID-19.

44. A method for treating a SARS-related betacoronavirus infection in a subject comprising, administering to the subject the pharmaceutical composition of any one of embodiments 35-42.

### EXAMPLES

### Example 1. Pseudotyped Lentivirus

We have developed a pseudotyped lentivirus expressing the SARS-CoV-2 glycoprotein. The pseudotyped lentivirus eliminates the need to perform direct work with SARS-CoV-2 while providing a method of analyzing SARS-CoV-2 spike (S) protein interaction with ACE2.

### Example 2. SARS-CoV-2 Inhibitor Screen

A lentivirus encoding a green fluorescent protein (GFP) that is pseudotyped with the SARS-CoV-2 surface glycoprotein responsible for ACE2 recognition is used to analyze disruption of binding of SARS-CoV-2 to ACE2-expressing cells.

A) Primary airway epithelia from humans are cultured at the air liquid interface (ALI) (Kuan et al. “Attenuated Amiloride-Sensitive Current and Augmented Calcium-Activated Chloride Current in Marsh Rice Rat (Oryzomys palustris) Airways.” iScience 2019, 19:737-748 and Reznikov et al. “Sex-specific airway hyperreactivity and sex-specific transcriptome remodeling in neonatal piglets challenged with intra-airway acid.” Am J Physiol Lung Cell Mol Physiol 2019, 316:L131-L143). Other ACE2-expressing cells can also be used.

B) Lentiviral Production. Lentiviral strategies similar to those described Overman et al. 2012 (“A role for ephrin-A5 in axonal sprouting, recovery, and activity-dependent plasticity after stroke.” Proc Natl Acad Sci USA 2012, 109:E2230-2239) are used. GFP enable the visualization of transduced cells. The pseudotyped lentivirus is added with the ACE2 expressing cells and incubated for a period time sufficient to allow binding of the pseudotyped lentivirus to the cells and expression of the virus-encoded GFP in the cells. Other pseudotyped viruses can also be used, including, but not limited to, adeno-associated virus (AAV). Binding of the SARS-CoV-2 spike protein can also be analyzed using recombinant proteins and ELISA-based methods.

C) Drug treatment. Clinically relevant doses of drugs are used when applicable. Duration of pretreatment is dependent upon the properties of the drug, including potential catalysis and stability.

D) Microscopy. Cultures are fixed with paraformaldehyde and fluorescence intensity quantified using imageJ. The number of GFP+ cells is normalized to the total number of cells.

Two time points are examined: drug pretreatment and drug treatment at the time of infection. Appropriate vehicles controls are used. The fraction of cells that express GFP compared to the total number of cells for each condition are then quantified. The fraction of total cells that express GFP is used an indicator of infection. Disruption of the binding of pseudotyped lentivirus to ACE2 indicates the molecule is effective in preventing viral entry into ACE2-expressing cells.

All analyses are run using GraphPad Prism at a 5% significance level. Differences are analyzed using a paired t-test (±drug treatment) for each drug.

Using this data and correlating regions of interaction of binding of molecules with ACE2 with inhibition of SARS-CoV-2 spike protein binding to ACE2 is then used to guide further development of SARS-CoV-2 therapeutics. In other words, molecules having the strongest inhibitory effect on ACE2-SARS-CoV-2 spike protein interaction can be used to determine the region(s) of ACE2 whose blockage (such as steric blockage) or disruption most interfere with SARS-CoV-2 binding and infection. Molecules specifically targeting those regions of ACE2 are then developed and/or analyzed for use as SARS-CoV-2 therapeutics.

Compounds identified in this assay are further tested against live SARS-CoV-2 virus one or more or cell culture ACE-2 expressing mammalian cells, primary airway epithelia cells, animal models, or humans.

### Example 3. Molecular Docking Analysis

Molecular docking analysis was used to identify molecules predicted to disrupt binding of the SARS-CoV-2 spike protein to ACE2 (see FIG. 1). Seven drugs were identified through molecular docking analysis. These molecules are expected to act as competitive and non-competitive antagonists that prevent binding of SARS-Cov-2 to ACE2. One of the drug candidates is the active ingredient, cetirizine, found in the allergy medicine, ZYRTEC. Another drug candidate is the compound diminazene that we identified as being protective in alleviating airway obstruction in a porcine model of airway injury. It is expected that drugs known to bind and/or effect activity of ACE2 and alter or block the identified ACE2 SARS-CoV-2 spike protein interaction site will inhibit SARS-CoV-2 binding to ACE2, thereby inhibiting infection (i.e., decreasing viral infectivity) or reducing the effects of infection.

It is expected that both pretreatment and co-treatment will have a beneficial effect.

### Example 4. Therapeutic Anti-SARS-CoV-2 Monoclonal Antibodies

Structural analysis of the interface between ACE2, based on PDB code 6M17, was used to identify a region of the SARS-CoV-2 spike protein suitable for use as an epitope in generating monoclonal antibodies useful for blocking interaction between the SARS-CoV-2 spike protein and ACE2 (see FIG. 1). In some embodiments, the monoclonal antibody binds to one or more amino acids in the region of amino acids 492-503 of the SARS-CoV-2 coronavirus spike protein.

### Example 5. Therapeutic Anti ACE2 Monoclonal Antibodies

Structural analysis of the interface between ACE2, based on PDB code 6M17, was used to identify a region of the ACE2 protein suitable for use as an epitope in generating monoclonal antibodies useful for blocking interaction between the SARS-CoV-2 spike protein and ACE2 (see FIG. 1). In some embodiments, the monoclonal antibody binds to one or more amino acids in the region of amino acids 31-42 of ACE2.

### Example 6. Diminazene Aceturate Prevents Airway Obstruction in a Porcine Model of Airway Injury

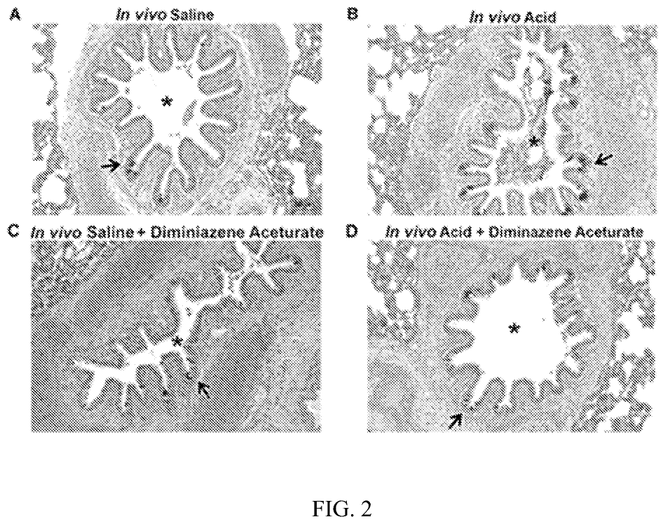

Diminazene binds to ACE2 and is predicted to inhibit binding of SARS-CoV-2 to ACE2. Air flow limitation in acute respiratory distress syndrome (ARDS) is attributed to obstruction of the small airways (Morales et al. “Small airway remodeling in acute respiratory distress syndrome: a study in autopsy lung tissue.” Crit Care 2011, 15:R4). Using a porcine model of airway injury, it was shown that diminazene aceturate attenuated airway obstruction (Liao et al. “Acid exposure disrupts mucus secretion and impairs mucociliary transport in neonatal piglet airways.” Am J Physiol Lung Cell Mol Physiol 2020) (FIG. 2). Further, diminazene aceturate has been shown to bind to AT rich nucleotide sequences and prevent plasmid DNA replication (Rajewska et al. “AT-rich region and repeated sequences—the essential elements of replication origins of bacterial replicons.” FEMS Microbiol Rev 2012, 36:408-434). Thus, treatment of a subject with diminazene aceturate might have the added benefit of improving disease symptoms as well as decreasing viral replication.

### Example 7. Identification of SARS-CoV-2 Therapeutics and Combination Therapeutics

Preclinical Step 1: COVID-19 and receptor binding candidate drugs. Toxicity and dosage information is available for 1,345 FDA approved drugs (www.epa.gov/nheerl/dsstox/). These drugs can be more rapidly translated into clinical trials compared to new candidates.

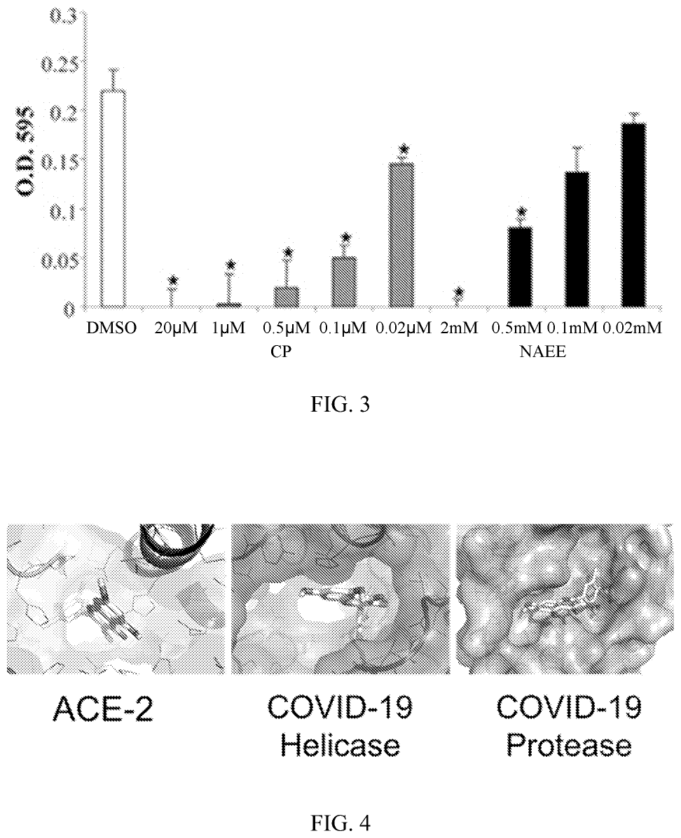

Solved high-resolution crystal structures of the apo-bound and inhibitor-bound forms of ACE2 have provided the basis for strategies to identify ACE2 inhibitors and compounds that block SARS coronavirus spike protein-mediated cell fusion. We used a structure-based selection strategy to screen >100K library of drug-like small molecules and identified an ACE2 inhibitor, N-(2-Aminoethyl)-1-aziridineethanamine (NAEE), that inhibits SARS S-glycoprotein-mediated cell fusion (FIG. 3). These data suggest that NAEE, and potentially other ACE2 inhibitors, may inhibit COVID-19 binding. Since NAEE is not an approved drug, it is not suitable for rapid translation into clinical trials.

Next, we screened 1,207 FDA approved drugs for ACE2 binding by molecular docking and identified drugs that could potentially be used for modulation of ACE2 interactions with the COVID-19 spike protein (Table). For example, hydroxyzine is an approved drug that inhibited ACE2 activity. These data suggest that hydroxyzine, and other ACE2 binding FDA approved drugs, may inhibit coronavirus spike protein interactions. Since the active metabolite of hydroxyzine is cetirizine (Zyrtec), these drugs represent candidates for treating or inhibiting SARS-CoV-2 infection.

TABLE 2

| Approved Drug | ACE2 binding activity |
|---|---|
| Labetalol | Enhanced velocity of ACE2 |
| Diminazene | Enhanced velocity of ACE2 |
| Aprindine | Inhibited velocity of ACE2 |
| Minithixen | Inhibited velocity of ACE2 |
| Hydroxyzine (Atarax) | Inhibited velocity of ACE2 |
| Cetirizine (Zyrtec) | Active metabolite of hydroxyzine |
| Triethylenetetramine | Similar to SARS/ACE2 inhibitor NAEE |

FDA approved drugs of three general types are selected for analysis: (1) ACE2 binding drugs, 2) antiviral drugs and (3) coronavirus replication inhibitors (e.g., protease, helicase binding, FIG. 4). Drugs are tested for antiviral activity and cytotoxicity, singly and in combination.

### Example 8. The Receptor Binding Domain of the Surface Spike Glycoprotein of SARS-CoV-2 Binds the Open Conformation of ACE2

Inhibitor binding favors the closed conformation of ACE2, which reduces interactions with the SARS spike proteins CoV and CoV-2. We previously defined a compound that binds ACE2 and inhibited contact with the SARS-CoV spike protein. Substrate binding site inhibitor N-(2-aminoethyl)-1 aziridine-ethanamine) inhibited ACE2 interactions with SARS-CoV spike protein.

Approach:

Measuring effects of ACE2 binding drugs on COVID-19/SARS-CoV-2 spike protein interactions.

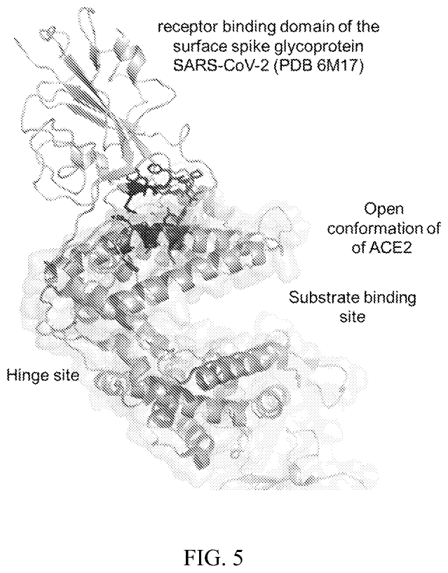

FIG. 5 illustrates amino acids at the interface or ACE2 and the SARS-CoV-2 spike protein interaction. Antibodies that bind to epitopes in these regions are expected to interfere with the spike protein binding to ACE2, thereby preventing fusion and infection.

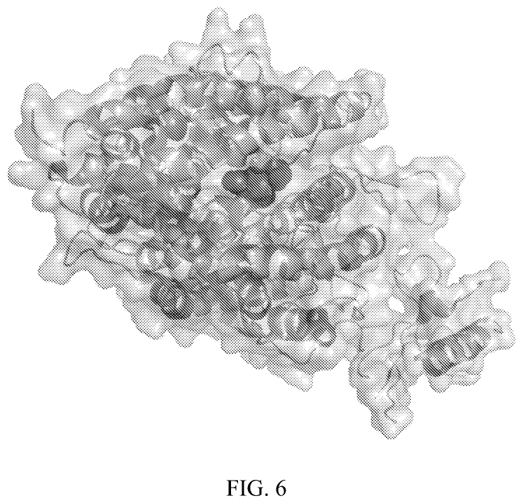

FIG. 6 illustrates the closed conformation of ACE2 with drug in the active site

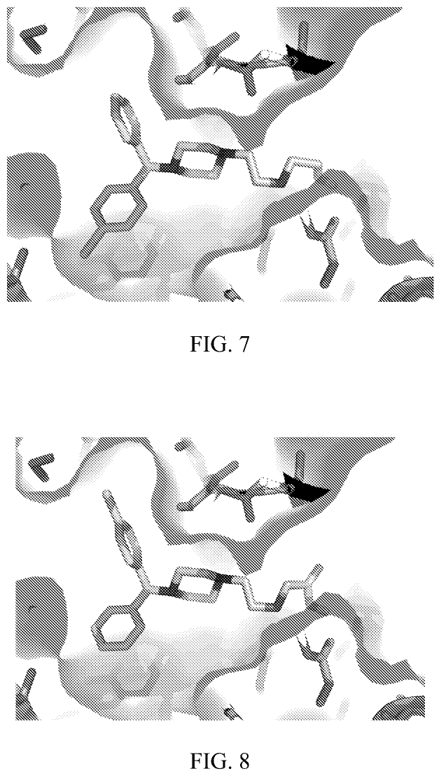

FIG. 7 illustrates binding to ACE2 posed by molecular docking. ΔG estimate=—8.6 kcal per mol.

FIG. 8 illustrates Dextrocetirizine binding to ACE2 posed by molecular docking. ΔG estimate=—8.8 kcal per mol.

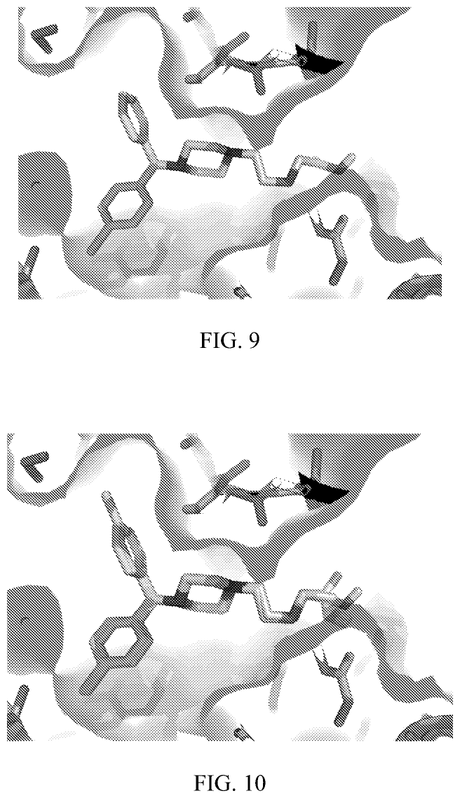

FIG. 9 illustrates Levocetirizine binding to ACE2 posed by molecular docking. ΔG estimate=—8.6 kcal per mol.

FIG. 10 illustrates an overlay image of Hydroxyzine, Dextrocetirizine, and Levocetirizine binding to ACE2.

### Example 9. Inhibition of SARS-CoV-2 Viral Entry into Host Cells

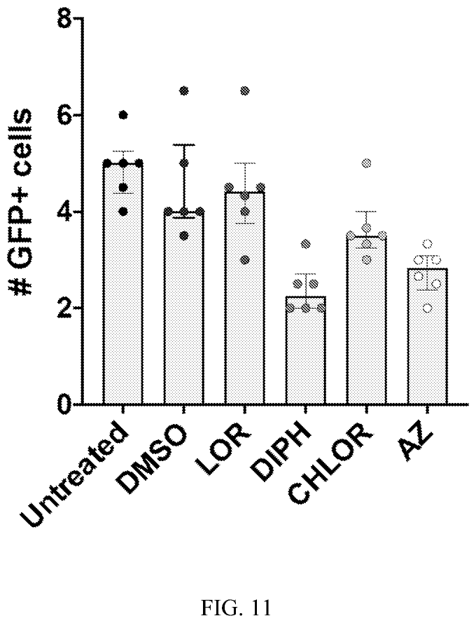

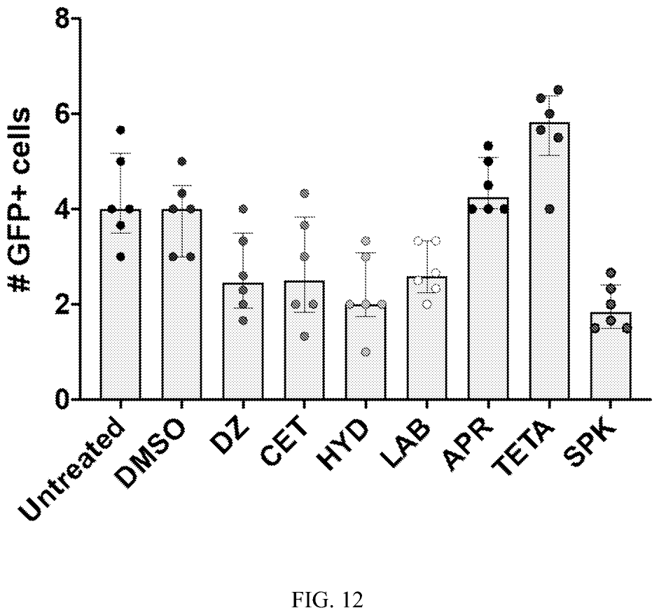

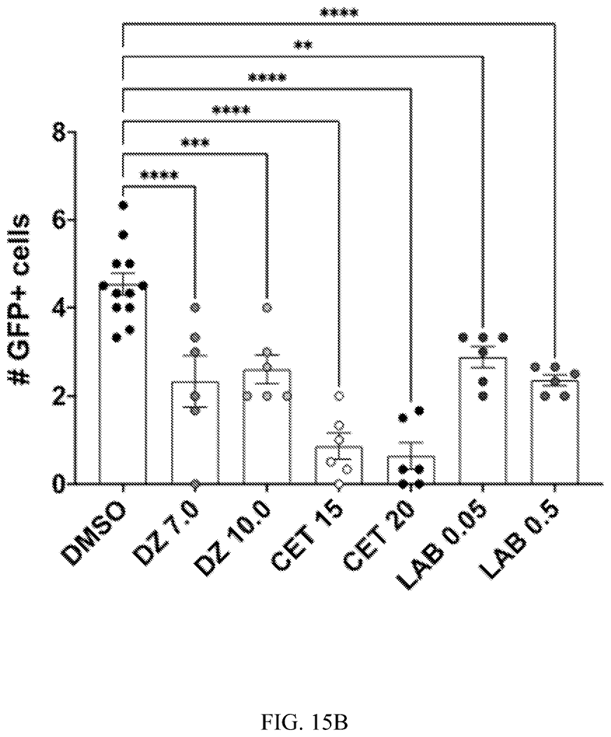

A lentivirus system pseudotyped to replace the surface glycoprotein with the full length SARS-CoV-2 spike glycoprotein was used to test interaction of SARS-CoV-2 spike protein with host cells. The pseudovirus expressed GFP. In this system, infected cells express the Green Fluorescent Protein (GFP). Drugs that decrease viral entry are expected to reduce the number of GFP⁺ cells. The number of GFP positive cells per image field was determined 72 hours after a single application of drugs in HEK293T cells overexpressing human ACE2. A decrease in GFP suggests the drugs disrupted the ability of the SARS-CoV-2 spike protein to interact with ACE2. Diminazene (DZ), Diphenhydramine (DIPH), Cetirizine (CET), Loratadine (LOR), Labetalol (LAB), Chlorpheniramine (CHLOR), Hydroxyzine (HYD), Azelastine (AZ), Aprindine (APR), and triethylenetetramine (TETA) were each dissolved in DMSO. Engineered lentivirus expressing the SARS-CoV-2 spike glycoprotein and the Green Fluorescent Protein (GFP) were incubated with ACE2 expressing airway epithelial cells and DMSO or drug. As shown in FIGS. 11 and 12, diphenhydramine (DIPH), azelastine (AZ), and hydroxyzine substantially reduced viral infection. The data in FIG. 11 were obtained with drug concentrations of LOR, 10 μg/ml; DIPH, 25 μg/ml; CHLOR, 8 μg/ml; and AZ, 7 μg/ml. Chlorpheniramine (CHLOR), diminazene (DIA), Azelastine (AZ), Diminazene (DZ), Cetirizine (CET), Hydroxyzine (HYD), and Labetalol (LAB) also reduced viral infection. FIG. 15A-B, shows efficacy of varying concentrations of the drugs in inhibiting viral entry into cells. Numbers after the drug name indicate concentrations of the drug in μg/ml. As shown in FIG. 15A, hydroxyzine, azelastine, and labetalol significantly lowered the level of SARS-CoV-2 pseudovirus entry in ACE2 expressing cells, even at sub μg/mL concentrations. As shown in FIG. 15A, diminazene, cetirizine, and labetalol each significantly lowered the level of SARS-CoV-2 pseudovirus entry in ACE2 expressing cells.

### Example 10. Effect of Hydroxyzine on Viral Entry into Host Cells

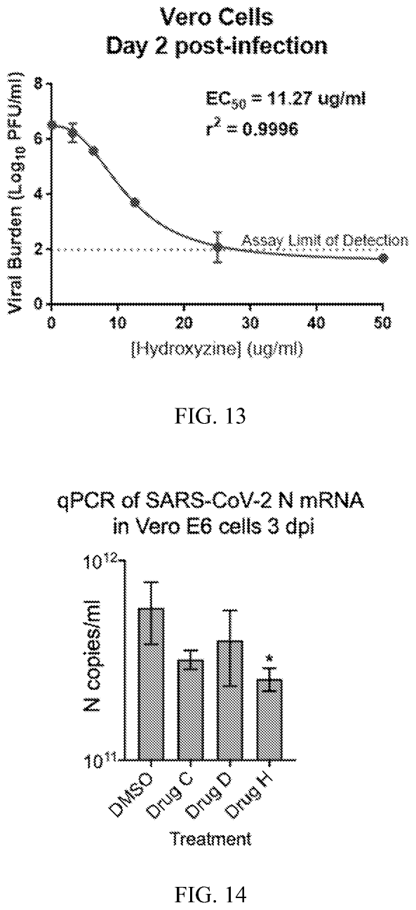

The effect of hydroxyzine a SARS-CoV-2 isolate infection of Vero E6 cells was analyzed. SARS-CoV-2 virus (isolate USA-WA1/2020, GenBank MN985325.1) was incubated with Vero E6 in the presence of varying concentrations of hydroxyzine. Viral burden in the cells was measures two days post-infection by plaque assay on Vero E6 cells. As shown in FIG. 13, hydroxyzine lowered viral burden to below the level of detection, with an EC50 of 11.27 μg/mL. Myricetin was observed to have in vitro activity SARS-CoV-2 (data not shown).

### Example 11. Effect of Cetirizine, Diminazene, and Hydroxyzine on Viral Activity

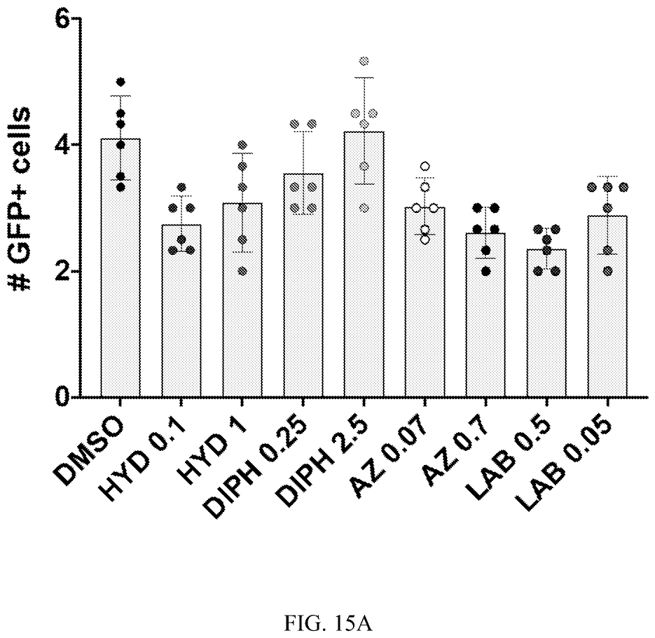

Antiviral activity of cetirizine, diminazene, and hydroxyzine were measured using an independent SARS-CoV-2 isolate. Vero E6 cells with infected with the human SARS-CoV-2 isolate USA-UF-1/2020 (GenBank MT295464) in the presence or absence of cetirizine (C), diminazene (D), or hydroxyzine (H). SARS-CoV-2 RNA was measured in each sample by quantitative PCR of infected Vero E6 cells. As shown in FIG. 14, hydroxyzine significantly lowered the level of SARS-CoV-2 mRNA expressed in the cells.

The data show that diphenhydramine, azelastine and hydroxyzine showed efficacy against SARS-CoV-2 infection in vitro.

### Example 12. Diphenhydramine, Chlorpheniramine and Azelastine Usage Correlate with Protection from SARS-CoV-2 Infection

| | | Lower | Upper | | | | | | |
|---|---|---|---|---|---|---|---|---|---|
| | Odds | confidence | confidence | | | | | | |
| Drug | ratio | interval | interval | p value | A | B | C | D | Drug class |
| Hydroxyzine | 0.76 | 0.44 | 1.23 | 0.323 | 18 | 358 | 553 | 8349 | Prescription |
| Brompheniramine | 0.76 | 0.02 | 4.78 | 1 | 1 | 20 | 570 | 8687 | OTC |
| Cetirizine | 0.73 | 0.38 | 1.28 | 0.315 | 13 | 269 | 558 | 8438 | OTC |
| Fexofenadine | 0.69 | 0.08 | 2.66 | 1 | 2 | 44 | 569 | 8663 | OTC |
| Loratadine | 0.59 | 0.31 | 1.03 | 0.067 | 13 | 330 | 558 | 8377 | OTC |
| Diphenhydramine | 0.38 | 0.26 | 0.54 | 6.05 × 10⁻¹⁰ | 35 | 1277 | 536 | 7430 | OTC |
| Levocetirizine | 0.35 | 0.01 | 2.09 | 0.522 | 1 | 43 | 570 | 8664 | Prescription |
| Chlorpheniramine | 0.23 | 0.06 | 0.59 | 0.00038 | 4 | 261 | 567 | 8446 | OTC |
| Azelastine | 0.16 | 0.02 | 0.57 | 0.00073 | 2 | 193 | 569 | 8514 | Prescription |

These data demonstrate that diphenhydramine, chlorpheniramine and azelastine usage correlated with protection from SARS-CoV-2 infection in this UCSF Health population in a statistically significant manner. Diphenhydramine usage, in particular, showed a strong p value of 6.05×10⁻¹⁰ for protection from SARS-CoV-2 infection.

### Example 12. Antiviral Activity of Specific Compounds Against SARS-CoV-2 Isolates In Vitro

The indicated drugs were tested for the ability to disrupt SARS-CoV-2/ACE2 interactions using a lentivirus pseudotyped to express the SARS-CoV-2 spike protein using recombinant spike protein and the DMSO vehicle as positive and negative controls, respectively. Drugs were also tested for direct antiviral activity by measuring effects on infection of Vero E6 cells with SARS-CoV-2 isolate USA-UF-1/2020.

Vero E6 cells were plated in 6 well plates with replicates on different plates. Monolayers were infected with virus master mix at 100 PFU/ml aliquoted in separate tubes with drugs at designated concentrations for 1 h. Monolayers were overlaid with MEM in 1.5% low-melt agarose with drugs at concentrations indicated above. Plaques were enumerated at 72 h post infection. For dose response curves, virus was diluted and allowed to infect Vero E6 across the indicated drug concentrations in DMEM+10% FBS in triplicate for 1 h with gentle rocking every 10 min. Solution was removed, then cells overlain with MEM+5% FBS in 1.5% low melt agarose containing the indicated drug concentrations and incubated at 37° C. with 5% CO2 for 3 dpi. 200 μl PBS containing 0.03% neural red was added to wells and plaques counted 3-6 h later to determine apparent PFU/ml.

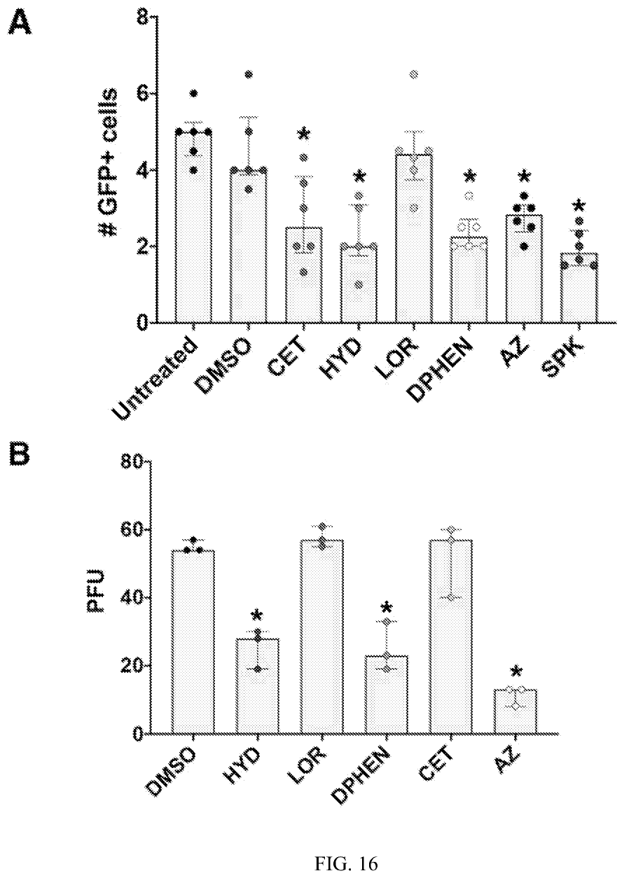

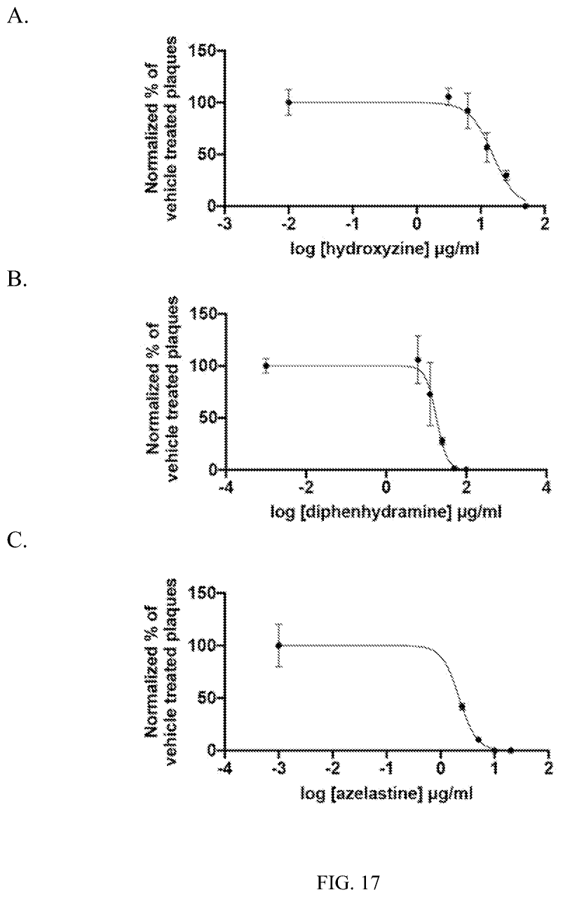

An engineered GFP-expressing lentivirus pseudotyped with the SARS-CoV-2 surface glycoprotein was used to infect ACE2 expressing HEK293 cells in the presence and absence of DMSO (control), 10 μg/mL cetirizine (CET), 10 μg/mL hydroxyzine (HYD), 1.5 μg/mL loratadine (LOR), 25 μg/mL diphenhydramine (DIPH), 7.0 μg/mL azelastine (AZ), or 13. ng/mL spike protein control (SPK). The number of GFP positive cells per image field 72 h after a single application of drugs in HEK293T cells overexpressing human ACE2 is shown in FIG. 16A (n=6 independent experiments. *p=0.0044 DMSO vs CET *; p=0.0002 DMSO vs HYD; *p=0.0006 DMSO vs DIPH; *p=0.0052 DMSO vs AZ; p<0.0001 DMSO vs SPK; Data were analyzed by a one-way ANOVA followed by a Dunnett multiple comparison test. In panel FIG. 16, the median+interquartile range are shown). The numbers of SARS-CoV-2 isolate USA-UF-1/2020 viral plaques present in Vero E6 cells 3 dpi following exposure to DMSO negative control, HYD, LOR, DIPHEN, CET, and AZ is shown in FIG. 16B (n=3 independent experiments. *p=0.0003 DMSO vs HYD *; p=0.0003 DMSO vs DIPH; *p<0.0001 DMSO vs AZ.) Dose response curves against SARS-CoV-2 isolate USA-UF-1/2020 in Vero E6 cells for hydroxyzine (A), diphenhydramine (B) and azelastine (C) are shown in FIG. 17A-C (n=3 in dependent experiments). Hydroxyzine, diphenhydramine and azelastine exhibited direct antiviral activity against SARS-CoV-2 isolates in vitro.

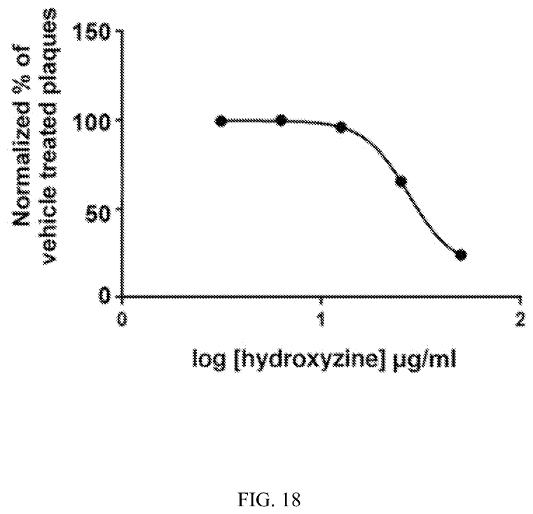

Drug susceptibility assays were performed for hydroxyzine against the USA-WA1/2020 strain of SARS-CoV-2 on human lung A549 cells that were transfected with hACE2 (ACE2-A549) cells. For these studies, ACE2-A549 cells were seeded into 6 well plates and allowed to attach overnight at 37 C, 5% CO2. Cells were pre-treated with various concentrations of hydroxyzine for 4 h prior to infection. Confluent cell monolayers were then infected with USA-WA1/2020 at a multiplicity of infection (MOI) equivalent to 0.03 PFU/cell and permitted to adsorb for 1 h. Plates were shaken every 15 min to ensure even distribution of the virus. Unbound virus was removed by washing cell monolayers twice with warm PBS and medium containing various concentrations of hydroxyzine were added to each well of the 6-well plate. One well served as a no-treatment control. Cell supernatants were harvested on day 4 post-inoculation, the day in which peak viral burden is achieved for this MOI on ACE2-A549 cells, clarified by high-speed centrifugation, and frozen at −80° C. until the end of the study. Assays were conducted in triplicate in two independent experiments. Dose response curve against SARS-CoV-2 isolate USA-WA1/2020 in human lung A549 cells for hydroxyzine (F) is shown in FIG. 18 (n=2 independent experiments).

Hydroxyzine, diphenhydramine and azelastine exhibited direct antiviral effects against SARS-CoV-2 in vitro. Drug concentrations effective for 50% inhibition of SARS-CoV-2 isolate USA-UF-1/2020 infection (EC50) of Vero E6 cells were 15.3 ng/ml for hydroxyzine, 17.4 ng/ml for diphenhydramine, and 2.24 ng/ml for azelastine. The drug concentration effective for 50% inhibition (EC50) for hydroxyzine against another SARS-CoV-2 isolate USA-WA1/2020 in the human lung cell line A549 overexpressing ACE2 was estimated to be 27.34 ng/ml. These data show that specific antihistamine exhibit antiviral effects on distinct isolates of SARS-CoV-2 in multiple cell types in vitro.

### Example 13. Synergy of Azelastine Plus Diphenhydramine and Diphenhydramine Plus Promethazine

N protein copies as a marker of SARS-CoV-2 replication were measured by qPCR 48 h after infection of VeroE6 cells with SARS-CoV-2 at an MOI of 0.01. Cells were treated with 5 μg/azelastine (AZ), 25 μg/mL diphenhydramine (DIPHEN), 15 ng/mL promethazine, 5 ng/mL azelastine plus 25 ng/mL diphenhydramine (AZ/DIPHEN), or 25 ng/mL diphenhydramine plus 15 ng/mL promethazine (DIPHEN/PMZ).

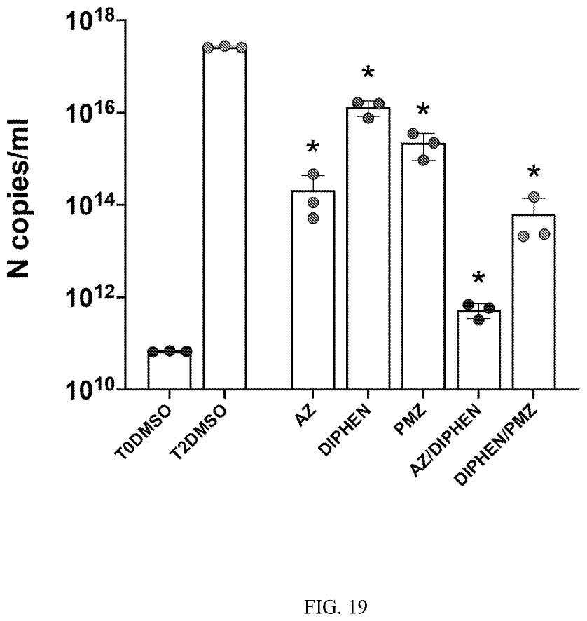

Azelastine plus diphenhydramine and diphenhydramine plus promethazine exhibited synergistic activity in vitro; reducing detected copies of viral N protein RNA ˜500× below azelastine alone levels (˜25,000 times below diphenhydramine alone). Promethazine by itself was able to reduce viral replication by ˜100× in vitro. Diphenhydramine and promethazine in combination reduced replication by ˜4,000× compared to untreated controls (FIG. 19).

Promethazine can be formulated with diphenhydramine or can be provided together with diphenhydramine in a separate formulation of pharmaceutical composition. Promethazine can be provided as an oral formulation (e.g., syrup or tablet), in as in injectable solution (such as for intramuscular injection).

### Example 14. Antibodies that Bind LQSYGFQPTNGV Peptide are SARS-CoV-2 Neutralizing Antibodies

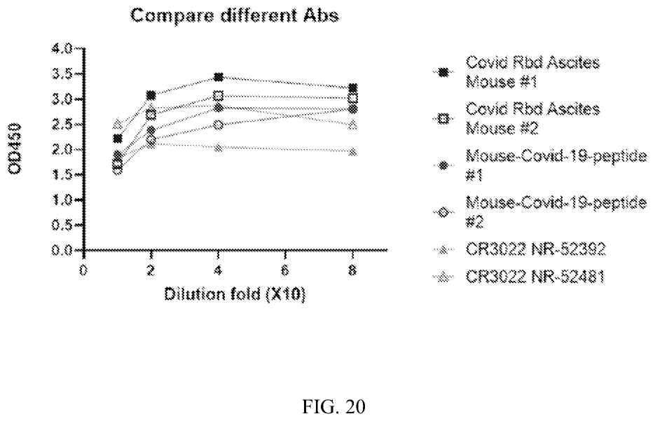

LQSYGFQPTNGV peptide was coupled to a carrier protein (MBS-KLH) and used to immunize mice. The peptide induced a strong immune response in the mice. Further, the mice produced anti-SARS-CoV-2 receptor binding domain (RBD) antibodies that inhibited ACE2-SARS-CoV-2 interaction. Using a competitive ELISA, it was found that antibodies from ascites of the peptide-immunized mice blocked interaction between host ACE2 and SARS-CoV-2 spike protein (FIG. 20). This data indicated antibodies produced from the peptide-immunized mice strongly inhibit ACE2 and SARS-CoV-2 RBD interaction. The data also indicates that the peptide itself inhibits ACE2 and SARS-CoV-2 RBD interaction. Thus, the LQSYGFQPTNGV peptide can use used to vaccinate against SARS-CoV-2 infection, antibodies against the LQSYGFQPTNGV peptide can be used to treat SARS-CoV-2 infection, and the LQSYGFQPTNGV peptide can be used as a therapeutic to treat SARS-CoV-2 infection.

### Example 15. Peptide Vaccination Provides Protection Against SARS-CoV-2 Infection

LQSYGFQPTNGV peptide was mixed with various adjuvants and injected intramuscularly into hACE2-transgenic mice. Mice were given a boost injection with the same composition one week after the initial (prime injection).

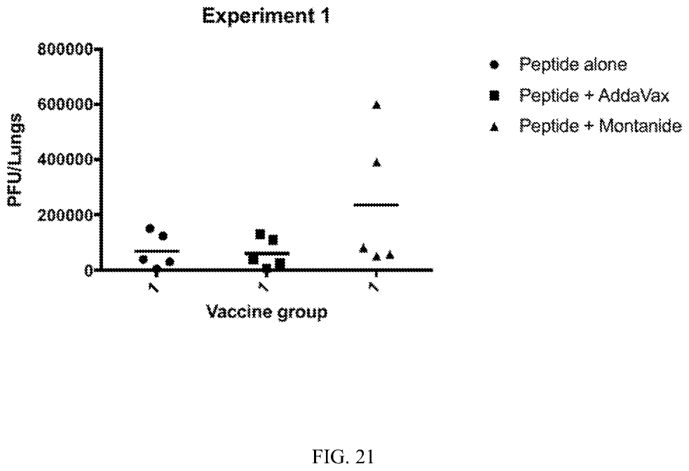

One week following the boost injection, hACE2 mice were inoculated intranasally with the SARS-CoV-2 stock virus (SARS-CoV-2 Strain WA-1) with a dosage of 10⁵ fifty-percent tissue culture infective dose (TCID₅₀). Body weights were recorded daily for all infected mice for 5 continuous days. The mice were sacrificed at 5 days post infection (dpi), and the lungs were collected for viral load analysis. All the mice in the control group vaccinated with PBS injection died within 5 dpi. All the mice in the peptide alone or peptide/adjuvant injected groups survived 5 dpi. Based on gross necropsy, mice vaccinated with peptide plus AddaVax adjuvant had normal-looking lungs. Mice vaccinated with peptide alone or peptide plus Montanide adjuvant had some damage/abscesses in the lungs. Mice immunized with peptide alone or with peptide plus AddaVax had low plaque forming units per liter in the lungs (FIG. 21). This data suggests the peptide can be used as a vaccine to provide strong protection against SARS-CoV-2 infection. Combining the peptide with an adjuvant can further increase the efficacy of the peptide as a vaccine.

1. A method of treating a subject suffering from infection by or susceptible to infection by a SARS-CoV-related betacoronavirus comprising administering to a subject a therapeutically effective amount of one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; myricetin; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

2. A method of treating a subject suffering from a SARS-CoV-related betacoronavirus-related illness comprising administering to the subject a therapeutically effective amount of one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; myricetin; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

3. A method of preventing infection by a SARS-CoV-related betacoronavirus comprising administering to a subject a therapeutically effective amount of one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; myricetin; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

4. The method of any one of claims 1-3, wherein the agent comprises diphenhydramine and/or azelastine.

5. The method of claim 4, wherein the diphenhydramine and/or azelastine comprises diphenhydramine.

6. The method of claim 5 wherein the method further comprises administering promethazine to the subject.

7. The method of claim 4, wherein the diphenhydramine and/or azelastine comprises azelastine.

8. The method of claim 4, wherein the diphenhydramine and/or azelastine comprises a combination of diphenhydramine and azelastine.

9. The method of any one of claims 1-3, wherein the agent comprises diminazene.

10. The method of any one of claims 1-3, wherein the agent comprises hydrozyzine.

11. The method of any one of claims 1-3, wherein the agent comprises cetirizene

12. The method of any one of claims 1-3, wherein the agent comprises chlorpheniramine.

13. The method of any one of claims 1-3, wherein the agent comprises labetalol.

14. The method of any one of claims 1-3, wherein the agent comprises the anti-ACE2 monoclonal antibody, or epitope binding fragment thereof, that binds to one or more amino acids in the region of amino acids 31-42 of ACE2 and inhibits binding of SARS-CoV-2 spike protein to ACE2.

15. The method of any one of claims 1-3, wherein the agent comprises the anti-spike protein monoclonal antibody, or epitope binding fragment thereof, that binds to one or more amino acids in the region of amino acids 492-503 of the SARS-CoV-2 coronavirus spike protein and inhibits binding of SARS-CoV-2 spike protein to ACE2.

16. The method of any one of claims 1-3, wherein the agent comprises the peptide have the amino acid sequence of SEQ ID NO: 1

17. The method of any one of claims 1-13, wherein the method further comprises administering one or more additional therapeutic agents.

18. The method of claim 8, wherein the one or more additional therapies comprises an effective amount of at least one drug selected from the group consisting of: one or more agents selected from the group consisting of: diphenhydramine; azelastine; diminazene; hydroxyzine; cetirizine; labetalol; promethazine; aprindine; minithixen; triethylenetetramine; N-(2-aminoethyl)-1-aziridine-ethanamine; bepotastine; fexofenadine; loratadine; desloratadine; ipratropium; metoclopramide; domperidone; an anti-spike protein monoclonal antibody, or epitope binding fragment thereof, that binds to one or more amino acids in the region of amino acids 492-503 of the SARS-CoV-2 coronavirus spike protein; an anti-ACE2 monoclonal antibody, or epitope binding fragment thereof, that binds to one or more amino acids in the region of amino acids 31-42 of ACE2, and a peptide have the amino acid sequence of SEQ ID NO: 1.

19. The method of any one of claims 1-2 and 4-18, wherein the subject has tested positive for a SARS-CoV-related betacoronavirus, has been exposed to a SARS-CoV-related betacoronavirus, is suspected of having been exposed to a SARS-CoV-related betacoronavirus, is at risk of being exposed to a SARS-CoV-related betacoronavirus, is suffering from or diagnosed with a SARS-CoV-related betacoronavirus-related illness, or is suffering from acute lung injury due to a SARS-CoV-related betacoronavirus-related illness.

20. The method of any one of claims 1-19, wherein the SARS-CoV-related betacoronavirus is SARS-CoV-2.

21. The method of any one of claims 2 and 4-20, wherein the SARS-CoV-related betacoronavirus-related illness is COVID-19.

22. A SARS-CoV-related betacoronavirus therapeutic comprising an anti-spike protein monoclonal antibody, or epitope binding fragment thereof, that binds to one or more amino acids in the region of amino acids 492-503 of the SARS-CoV-2 coronavirus spike protein and inhibits binding of SARS-CoV-2 spike protein to ACE2.

23. The SARS-CoV-related betacoronavirus therapeutic of claim 22, wherein the anti-spike protein monoclonal antibody, or epitope binding fragment thereof, binds the polypeptide LQSYGFQPTNGV (SEQ ID NO: 1).

24. A SARS-CoV-related betacoronavirus therapeutic comprising an anti-ACE2 monoclonal antibody, or epitope binding fragment thereof, that binds to one or more amino acids in the region of amino acids 31-42 of ACE2 and inhibits binding of SARS-CoV-2 spike protein to ACE2.

25. The SARS-CoV-related betacoronavirus therapeutic of claim 24, wherein the anti-spike protein monoclonal antibody, or epitope binding fragment thereof, binds the polypeptide KFNHEAEDLFYQ (SEQ ID NO: 2).

26. Use of the SARS-CoV-related betacoronavirus therapeutic of any one of claims 22-25 in the treatment of a subject suffering from infection by a SARS-CoV-related betacoronavirus.

27. A method of treating SARS-CoV-2 infection in a subject comprising administering to the subject an antibody, or an epitope binding fragment thereof, specific for LQSYGFQPTNGV (SEQ ID NO: 1) or KFNHEAEDLFYQ (SEQ ID NO: 2)

28. A SARS-CoV-2 vaccine comprising a polypeptide having at least 80% sequence identity to the amino acid sequence of SEQ ID NO: 1 or a peptide comprising 9-15 amino acids differing by 0, 1, 2, or 3 amino acid substitutions, deletions, insertions, or combinations thereof from SEQ ID NO: 1.

29. The SARS-CoV-2 vaccine of claim 28, wherein the polypeptide is no more than about 100 amino acids in length.

30. The SARS-CoV-2 vaccine of claim 28, wherein the polypeptide amino acid sequence consists of SEQ ID NO: 1.

31. The SARS-CoV-2 vaccine of any one of claims 28-30, further comprising an adjuvant.

32. The SARS-CoV-2 vaccine of claim 31, wherein the adjuvant comprises: AddaVax, Montanide, alum, Sigma Adjuvant System, or Freund's adjuvant.

33. Use of the vaccine of any one of claims 28-32 to induce a protective immune response in a subject to prevent and/or treat a SARS-CoV-2 infection.

34. A method of inducing an immune response to SARS-CoV-2 in a subject comprising: administering to the subject the vaccine of any one of claims 28-32.

35. A pharmaceutical composition comprising one or more agents selected from the group consisting of: diphenhydramine; azelastine; diminazene; hydroxyzine; cetirizine; labetalol; aprindine; minithixen; triethylenetetramine; N-(2-aminoethyl)-1-aziridine-ethanamine; bepotastine; fexofenadine; loratadine; desloratadine; ipratropium; metoclopramide; domperidone; an anti-spike protein monoclonal antibody, or epitope binding fragment thereof, that binds to one or more amino acids in the region of amino acids 492-503 of the SARS-CoV-2 coronavirus spike protein; and an anti-ACE2 monoclonal antibody, epitope binding fragment thereof, that binds to one or more amino acids in the region of amino acids 31-42 of ACE2, and a peptide have the amino acid sequence of SEQ ID NO: 1, for use in the treatment of a SARS-related betacoronavirus infection.

36. The pharmaceutical composition of claim 35 wherein the composition comprises diphenhydramine and azelastine.

37. The pharmaceutical composition of claim 35 or claim 32 further comprising at least one pharmaceutically acceptable excipient.

38. The pharmaceutical composition of any one of claims 35-37, further comprising one or more additional therapeutic agents.

39. The pharmaceutical composition of claim 35, wherein the additional therapeutic agent is selected from the group consisting of: an antihistamine, an IL-6 inhibitor, sarilumab, siltuximab, tocilizumab, an H2 blocker, ranitidine, famotidine, nizatidine, roxatidine, lafutidine, lavoltidine, niperotidine, an anticoagulant, heparin, warfarin, rivaroxaban, dabigatran, apixaban, edoxaban, enoxaparin, fondaparinux, an antitussive, dextromethorphan, benzonatate, an antibiotic, azithromycin, clarithromycin, doxycycline, levofloxacin, amoxicillin, clavulanate, an IL-1 inhibitor, rilonacept, canakinumab, gevokizumab, a cytostatic drug, azathioprine, methotrexate, mycophenolate, a cytokine inhibitor, an immune modulator, infliximab, basiliximab, daclizumab, rapamycin, interferon β-1b, an antiviral agent, remdesivir, lopinavir, ritonavir, ribavirin, a glucocorticoid, beclomethasone, betamethasone, budesonide, cortisone, dexamethasone, hydrocortisone, methylprednisolone, prednisolone, prednisone, triamcinolone, a non-steroidal anti-inflammatory drug, phenazone, aspirin, celecoxib, diclofenac, ibuprofen, indomethacin, naproxen, oxaprozin, piroxicam, or a combination thereof.

40. The pharmaceutical composition of claim 39 wherein the pharmaceutical composition comprises diphenhydramine and promethazine.

41. The pharmaceutical composition of any of claims 35-40, wherein the pharmaceutical composition is formulated for oral delivery, intravenous injection, intramuscular injection, subcutaneous injection, or inhalation.

42. The pharmaceutical composition of claim 41, wherein the pharmaceutical composition is dispensed by a metered-dose inhaler, a dry powder inhaler, a nebulizer, or a soft mist inhaler.

43. The pharmaceutical composition of any one of claims 35-42, wherein the SARS-related betacoronavirus infection comprises COVID-19.

44. A method for treating a SARS-related betacoronavirus infection in a subject comprising, administering to the subject the pharmaceutical composition of any one of claims 35-42.


SEQUENCE LISTING (2 sequences)

SEQ ID NO: 1 - protein, 12 aa, SARS-CoV-2
LQSYGFQPTNGV

SEQ ID NO: 2 - protein, 12 aa, Homo sapiens
KFNHEAEDLFYQ